(19) United States
(12) Patent Application Publication
(10) Pub. No.: US 2023/0174991 A1
(43) Pub. Date: Jun. 8, 2023

(54) KDO-FREE PRODUCTION HOSTS FOR OLIGOSACCHARIDE SYNTHESIS

(71) Applicant: INBIOSE N.V., Zwijnaarde (BE)

(72) Inventors: Joeri Beauprez, Zwijnaarde (BE); Pieter Coussement, Zwijnaarde (BE); Gart Peters, Zwijnaarde (BE)

(21) Appl. No.: 17/904,196

(22) PCT Filed: Feb. 12, 2021
(86) PCT No.: PCT/EP2021/053498

§ 371 Date: Aug. 12, 2022

(30) Foreign Application Priority Data
Feb. 14, 2020 (BE) 20205094

(51) Int. Cl. C12N 15/52; C07K 14/245; C12N 1/20; C12N 9/10; C12P 19/02; C12P 19/04; C12P 19/18; C12P 19/26; C12P 19/28

(52) U.S. Cl. CPC C12N 15/52; C07K 14/245; C12N 1/20; C12N 9/1029; C12N 9/1048; C12P 19/02; C12P 19/04; C12P 19/18; C12P 19/26; C12P 19/28; C12Y 204/99001; C12R 2001/19

(57) ABSTRACT

This disclosure relates to the technical field of synthetic biology and metabolic engineering. More particularly, this disclosure relates to the technical field of fermentation of metabolically engineered microorganisms. This disclosure describes engineered micro-organisms that produce oligosaccharides that are free of KDO-lactose impurities and/or KDO-oligosaccharide impurities.

### CROSS-REFERENCE TO RELATED APPLICATIONS

This application is a national phase entry under 35 U.S.C. § 371 of International Patent Application PCT/EP2021/053498, filed Feb. 12, 2021, designating the United States of America and published as International Patent Publication WO 2021/160828 A1 on Aug. 19, 2021, which claims the benefit under Article 8 of the Patent Cooperation Treaty to Belgian Patent Application Serial No. 2020/5094, filed Feb. 14, 2020.

### TECHNICAL FIELD

This disclosure relates to the technical field of synthetic biology and metabolic engineering. More particularly, this disclosure relates to the technical field of fermentation of metabolically engineered microorganisms. This disclosure describes engineered micro-organisms that produce oligosaccharides that are free of KDO-lactose impurities and/or KDO-oligosaccharide impurities.

### STATEMENT ACCORDING TO 37 C.F.R. § 1.821(c) or (e) —SEQUENCE LISTING SUBMITTED AS PDF FILE WITH A REQUEST TO TRANSFER CRF FROM PARENT APPLICATION

Pursuant to 37 C.F.R. § 1.821(c) or (e), files containing a TXT version and a PDF version of the Sequence Listing have been submitted concomitant with this application, the contents of which are hereby incorporated by reference. The uncompressed TXT version of the Sequence Listing is 77 KB.

### BACKGROUND

Oligosaccharides can be produced via enzymatic, chemical as well as fermentative production. However, all of them have significant disadvantages. For instance, chemical synthesis requires many sequential chemical steps and enzymatic synthesis requires expensive precursors, whereas the fermentative process is still under heavy development. Nonetheless, the latter has the highest industrial production potential.

One problem observed with fermentative production of oligosaccharides, e.g., sialylated oligosaccharides, is that in some instances KDO-lactose and/or KDO-oligosaccharide is produced instead of the desired oligosaccharide.

Another problem that arises when adapting a microbial host for oligosaccharide synthesis is that an enormous burden is created on the cell wall integrity of the microbial host, since the host (partially) shifts its cell wall glycan synthesis machinery into the synthesis of the desired oligosaccharide. Both cell wall glycan synthesis and oligosaccharide synthesis need specific nucleotide-activated sugars and glycosyltransferases that transfer the monosaccharide building block from a nucleotide-activated sugar onto a growing nascent saccharide chain. Often both cell wall glycan synthesis and oligosaccharide synthesis conflict with each other for the same nucleotide-activated sugars and/or glycosyltransferases. As such, cells adapted for oligosaccharide synthesis are more vulnerable due to a weaker and/or altered cell wall composition, and, confer higher osmotic stress compared to the native microbial host.

The microbe's cytoplasm is usually hypertonic to its surrounding environment and the net flow of free water is into the microbial cell. Without a strong cell wall, the microbial cell would burst from the osmotic pressure of the water flowing into the cell. To protect the cell from the environment and osmotic lysis, microbial cells are coated with an impressive array of distinct glycan structures that comprise their cell wall (Campanero-Rhodes et al., Front Microbiol. 2020 (doi.org/10.3389/fmicb.2019.02909); Tra and Dube, Chem. Commun. (Camb.) 2014, 50(36), 4659-4673; Vollmer et al., FEMS Microb. Rev. 2008, 32(2), 149-167).

Gram-negative bacteria (e.g., Escherichia coli) have a cell wall that consists of a double membrane enclosing a peptidoglycan (PG) containing periplasmatic space. PG is a glycosidic structure being composed of alternating GlcNAc and MurNAc sugars that are cross-linked by short peptide bridges. The inner membrane (IM) or cytoplasmic membrane is a phospholipid bilayer whereas the outer membrane (OM) is asymmetrical. The OM of Gram-negative bacteria is also a lipid bilayer, but its surface exposed outer leaflet is composed of lipopolysaccharides or LPS. The outer membrane structure protects the bacteria against harsh environmental conditions and forms a barrier against numerous stress factors. LPS is a glycophospholipid consisting of an antigenic, variable-size, carbohydrate chain covalently linked to lipid A, the conserved hydrophobic region structurally defined as N,O-acyl beta-1,6-D-glucosamine 1,4′-bisphosphate. The hydrophobic anchor of LPS is a glucosamine-based phospholipid backbone called 2-keto 3-deoxy-D-manno-octulosonate (KDO)-LipidA. The biogenesis of KDO-LipidA involves nine enzymes and was first uncovered by Christian R. H. Raetz, hence is referred to as the Raetz pathway. The ΔKDO phenotype is lethal. This lethal ΔKDO phenotype can be suppressed by overexpression of mutant msbA as disclosed by Meredith et al. (ACS Chem. Biol. 2006, 1(1):33-42) and/or by overexpression of mutant YhjD as disclosed by Mamat et al. (Mol. Microbiol. 2008, 67(3): 633-648) and WO 2013/036756 and/or by overexpression of LpxL as disclosed by WO 2017/119628. Both Meredith et al. (ACS Chem. Biol. 2006, 1(1):33-42) and Mamat et al. (Mol. Microbiol. 2008, 67(3): 633-648) do not further modify the viable KDO KO strains for production of a compound of interest. Both WO 2013/036756 and WO 2017/119628 further modify the viable KDO KO strains for production of recombinant protein, yet, recombinant protein expression does not create a burden on the cell wall integrity nor on the cell's resistance to osmotic stress. However, the suppression as found in the prior art described above is only partially and leaves the cell with a lowered resistance toward osmotic stress, which, as explained above, would be problematic when this cell is adapted to produce oligosaccharide.

Removal of KDO-oligosaccharides is done using a wide range of techniques such as ultracentrifugation, two-phase extraction or affinity chromatography, but there is no universal removal technique, and all depends highly on the product type and application.

Drouillard et al. (Carbohydr. Res. 2010 Jul. 2; 345(10):1394-9) and WO2014153253 describe that a substantially KDO-lactose-free sialyllactose production can be obtained by fermentation of a sialyllactose producing micro-organism having a reduced expression of the sialyltransferase. This reduced expression was obtained by reducing the strength of the promoter or ribosome binding site, limiting transcription or translation of the transferase. WO2019/118829 overcomes the unwanted synthesis of KDO-lactose, as example of a KDO-oligosaccharide, by a microbial host by expressing specific exogenous lactose-utilizing sialyltransferase enzymes that have more preference for CMP-sialic acid than for CMP-KDO as a substrate.

Based on the above, it can be concluded that the production of the KDO-lactose, and in a broader sense also KDO-oligosaccharides, is due to the glycosyltransferase present in the production host having affinity to the CMP-KDO produced by the microbial cell naturally producing KDO. Such glycosyltransferases accept CMP-KDO thereby producing the KDO-oligosaccharide side product.

There is thus a need in the art to reduce the cost of downstream processing in respect of removal of KDO-oligosaccharide and to create an expression host to be used in such a biotechnological production process for the production of oligosaccharides. Such expression platform should offer a way of oligosaccharides production while minimizing the cost for purifications and elimination.

### BRIEF SUMMARY

Surprisingly, a microbial cell naturally synthesizing KDO and genetically modified for the production of oligosaccharide has been created, wherein the KDO biosynthesis of the cell is knocked out or rendered less functional, such that the cell no longer produces KDO-lactose and/or KDO-oligosaccharides as unwanted side-product.

Gram-negative microbial cells contain a pathway for the synthesis of 3-deoxy-D-manno-octulosonate, so called KDO. This is an integral part of the outer membrane structure and forms a part of the endotoxins in, e.g., the Enterobacteriaceae. The deletion of the synthesis of the endotoxins leads to cell death. On the other hand, the presence of CMP-KDO in the cell can interfere in other pathways and already proved to lead to unwanted impurities in fermentative sialylated oligosaccharide production. Taking away the KDO-synthesis will destabilize the cell wall and cells will grow slower.

In this disclosure, KDO synthesis in oligosaccharide production hosts have been reduced and eliminated wherein production of the oligosaccharide is not hampered significantly by the reduced cell growth when producing oligosaccharides. Oligosaccharides pose osmotic stress on the cell, which is mediated by the outer membrane, KDO synthesis knockouts reduce the resistance. Surprisingly, ample cell fitness was still maintained after deletion of the KDO biosynthesis route. No KDO moieties are transferred anymore on the oligosaccharide. By doing so, the purity of the oligosaccharide is increased, by reducing KDO based side products. Reduced KDO synthesis also further enhanced downstream processing of the oligosaccharide production as an additional purification step would no longer be needed.

### Definitions

The words used in this specification to describe the disclosure and its various embodiments are to be understood not only in the sense of their commonly defined meanings, but to include by special definition in this specification structure, material or acts beyond the scope of the commonly defined meanings. Thus, if an element can be understood in the context of this specification as including more than one meaning, then its use in a claim must be understood as being generic to all possible meanings supported by the specification and by the word itself.

The various embodiments and aspects of embodiments of the disclosure described herein are to be understood not only in the order and context specifically described in this specification, but to include any order and any combination thereof. Whenever the context requires, all words used in the singular number shall be deemed to include the plural and vice versa. Unless defined otherwise, all technical and scientific terms used herein generally have the same meaning as commonly understood by one of ordinary skill in the art to which this disclosure belongs. Generally, the nomenclature used herein and the laboratory procedures in cell culture, molecular genetics, organic chemistry and nucleic acid chemistry and hybridization described herein are those well-known and commonly employed in the art. Standard techniques are used for nucleic acid and peptide synthesis. Generally, enzymatic reactions and purification steps are performed according to the manufacturer's specifications.

In the drawings and specification, there have been described embodiments of the disclosure and, although specific terms are employed, the terms are used in a descriptive sense only and not for purposes of limitation, the scope of the invention being set forth in the following claims. It must be understood that the illustrated embodiments have been set forth only for the purposes of example and that it should not be taken as limiting the invention. It will be apparent to those skilled in the art that alterations, other embodiments, improvements, details and uses can be made consistent with the letter and spirit of the disclosure herein and within the scope of this disclosure, which is limited only by the claims, construed in accordance with the patent law, including the doctrine of equivalents. In the claims that follow, reference characters used to designate claim steps are provided for convenience of description only, and are not intended to imply any particular order for performing the steps.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated,” as the term is employed herein. Similarly, a “synthetic” sequence, as the term is used herein, means any sequence that has been generated synthetically and not directly isolated from a natural source. “Synthesized,” as the term is used herein, means any synthetically generated sequence and not directly isolated from a natural source.

The term “endogenous” within the context of the present disclosure refers to any polynucleotide, polypeptide or protein sequence that is a natural part of a cell and is occurring at its natural location in the cell chromosome. The term “exogenous” refers to any polynucleotide, polypeptide or protein sequence that originates from outside the cell under study and is not a natural part of the cell or that is not occurring at its natural location in the cell chromosome or plasmid.

“Recombinant” means genetically engineered DNA prepared by transplanting or splicing genes from one species into the cells of a host organism of a different species. Such DNA becomes part of the host's genetic make-up and is replicated. “Mutant” cell or microorganism as used within the context of the present disclosure refers to a cell or microorganism that is genetically engineered or has an altered genetic make-up.

The term “modified expression” of a gene relates to a change in expression compared to the wild-type expression of the gene in any phase of the production process of the oligosaccharide. The modified expression is either a lower or higher expression compared to the wild type, wherein the term “higher expression” is also defined as “overexpression” of the gene in the case of an endogenous gene; or “expression” or “overexpression” in the case of a heterologous gene that is not present in the wild-type strain. Lower expression or reduced expression or “genes that are rendered less-functional or non-functional” is/are obtained by means of common well-known technologies for a skilled person (such as the usage of siRNA, CRISPR, CRISPRi, riboswitch, recombineering, homologous recombination, ssDNA mutagenesis, RNAi, miRNA, asRNA, mutating genes, knocking-out genes, transposon mutagenesis, . . . ) that are used to change the genes in such a way that they are less-able (i.e., statistically significantly “less-able” compared to a functional wild-type gene) or completely unable (such as knocked-out genes) to produce functional final products. Next to changing the gene of interest in such a way that lower expression is obtained as described above, lower expression can also be obtained by changing the transcription unit, the promoter, an untranslated region, the ribosome binding site, the Shine Dalgarno sequence or the transcription terminator. Lower expression or reduced expression or “genes that are rendered less-functional or non-functional” may, for instance, be obtained by mutating one or more base pairs in the promoter sequence or changing the promoter sequence fully to a constitutive promoter with a lower expression strength compared to the wild type or an inducible promoter that result in regulated expression or a repressible promoter that results in regulated expression. Overexpression or expression is obtained by means of common well-known technologies for a skilled person, wherein, e.g., the gene is part of an “expression cassette” that relates to any sequence in which a promoter sequence, untranslated region sequence (containing either a ribosome binding sequence or Kozak sequence), a coding sequence (for instance, a membrane protein gene sequence) and optionally a transcription terminator is present, and leading to the expression of a functional active protein. The expression is either constitutive or conditional or regulated.

The term “riboswitch” as used herein is defined to be part of the messenger RNA that folds into intricate structures that block expression by interfering with translation. Binding of an effector molecule induces conformational change(s) permitting regulated expression post-transcriptionally.

The term “wild type” refers to the commonly known genetic or phenotypical situation as it occurs in nature.

The terms “cells” and “host cells” and “recombinant host cells,” which are used interchangeably herein, refer to cells that are capable of or have been transformed with a vector, typically an expression vector. The host cells used herein are cells naturally comprising KDO biosynthesis. Such cells can be Gram-negative bacterial cells, for example, Bacteriaceae. It is understood that such terms refer not only to the particular subject cell, but to the progeny or potential progeny of such a cell. Because certain modifications may occur in succeeding generations due to either mutation or environmental influences, such progeny may not, in fact, be identical to the parent cell, but are still included within the scope of the term as used herein.

The term “defective” as used herein, with regard to a gene or gene expression, means that the gene is not a wild-type gene and that the organism does not have a wild-type genotype and/or a wild-type phenotype. The defective gene, genotype or phenotype may be the consequence of a mutation in that gene, or of a gene that regulates the expression of that gene (e.g., transcriptional or post-transcriptional), such that its normal expression is disrupted or extinguished. “Disrupted gene expression” is intended to include both complete inhibition and decreased gene expression (e.g., as in a leaky mutation), below wild-type gene expression.

The terms “conditional,” “regulated,” “inducible,” or similar terms, refer, in particular, to gene expression that is not constitutive, but that takes place in response to a stimulus (e.g., temperature, heavy metals or other medium additive).

The term “nucleic acid” refers to polynucleotides or oligonucleotides such as deoxyribonucleic acid (DNA), and, where appropriate, ribonucleic acid (RNA). The term should also be understood to include, as equivalents, analogs of either RNA or DNA made from nucleotide analogs and as applicable to the embodiment being described, single (sense or antisense) and double-stranded polynucleotides.

As used herein, the term “transfection” means the introduction of a nucleic acid (e.g., via an expression vector) into a recipient cell by nucleic acid-mediated gene transfer.

“Transformation,” as used herein, refers to a process in which a cell's genotype is changed as a result of the cellular uptake of exogenous DNA or RNA. A transformed cell can also be one that expresses a nucleic acid that interferes with the expression of an endogenous nucleic acid.

As used herein, the term “transgene” means a nucleic acid that has been introduced into a cell. A transgene could be partly or entirely heterologous, i.e., foreign, to the transgenic cell into which it is introduced, or, can be homologous to an endogenous gene of the cell into which it is introduced, but is designed to be inserted, or is inserted, into the cell's genome in such a way as to alter the genome of the cell into which it is inserted. A transgene can also be present in a cell in the form of an episome.

The term “treating” a subject for a condition or disease, as used herein, is intended to encompass curing, as well as ameliorating at least one symptom of the condition or disease.

The term “vector” refers to a nucleic acid molecule that is capable of transporting another nucleic acid to which it has been linked. Vectors capable of directing the expression of genes to which they are operatively linked are referred to herein as “expression vectors.”

The term “expression system” as used herein refers to an expression vector under conditions whereby an mRNA may be transcribed and/or an mRNA may be translated into protein, structural RNA, or other cellular component. The expression system may be an in vitro expression system, which is commercially available or readily made according to art known techniques, or may be an in vivo expression system, such as a eukaryotic or prokaryotic cell containing the expression vector. In general, expression vectors of utility in recombinant DNA techniques are often in the form of “plasmids,” which refer generally to circular double-stranded DNA loops that, in their vector form, are not bound to the chromosome. In the present specification, “plasmid” and “vector” are used interchangeably as the plasmid is the most commonly used form of vector. However, the disclosure is intended to include such other forms of expression vectors that serve equivalent functions and are well known in the art or that become known in the art subsequently hereto (e.g., cosmid, phagemid and bacteriophage vectors).

The term “oligosaccharide” as used herein refers to a saccharide polymer containing a small number, typically two to fifteen, preferably three to fifteen, of simple sugars, i.e., monosaccharides. Examples of oligosaccharides include but are not limited to mammalian milk oligosaccharides, glycosaminoglycans, chitosans, chondrotoines, heparosans, glucuronylated oligosaccharides, fucosylated oligosaccharides, neutral oligosaccharides and/or sialylated oligosaccharides.

The term “monosaccharide” as used herein refers to saccharides containing only one simple sugar. Examples of monosaccharides comprise Hexose, D-Glucopyranose, D-Galactofuranose, D-Galactopyranose, L-Galactopyranose, D-Mannopyranose, D-Allopyranose, L-Altropyranose, D-Gulopyranose, L-Idopyranose, D-Talopyranose, D-Ribofuranose, D-Ribopyranose, D-Arabinofuranose, D-Arabinopyranose, L-Arabinofuranose, L-Arabinopyranose, D-Xylopyranose, D-Lyxopyranose, D-Erythrofuranose, D-Threofuranose, Heptose, L-glycero-D-manno-Heptopyranose (LDmanHep), D-glycero-D-manno-Heptopyranose (DDmanHep), 6-Deoxy-L-altropyranose, 6-Deoxy-D-gulopyranose, 6-Deoxy-D-talopyranose, 6-Deoxy-D-galactopyranose, 6-Deoxy-L-galactopyranose, 6-Deoxy-D-mannopyranose, 6-Deoxy-L-mannopyranose, 6-Deoxy-D-glucopyranose, 2-Deoxy-D-arabino-hexose, 2-Deoxy-D-erythro-pentose, 2,6-Dideoxy-D-arabino-hexopyranose, 3,6-Dideoxy-D-arabino-hexopyranose, 3,6-Dideoxy-L-arabino-hexopyranose, 3,6-Dideoxy-D-xylo-hexopyranose, 3,6-Dideoxy-D-ribo-hexopyranose, 2,6-Dideoxy-D-ribo-hexopyranose, 3,6-Dideoxy-L-xylo-hexopyranose, 2-Amino-2-deoxy-D-glucopyranose, 2-Amino-2-deoxy-D-galactopyranose, 2-Amino-2-deoxy-D-mannopyranose, 2-Amino-2-deoxy-D-allopyranose, 2-Amino-2-deoxy-L-altropyranose, 2-Amino-2-deoxy-D-gulopyranose, 2-Amino-2-deoxy-L-idopyranose, 2-Amino-2-deoxy-D-talopyranose, 2-Acetamido-2-deoxy-D-glucopyranose, 2-Acetamido-2-deoxy-D-galactopyranose, 2-Acetamido-2-deoxy-D-mannopyranose, 2-Acetamido-2-deoxy-D-allopyranose, 2-Acetamido-2-deoxy-L-altropyranose, 2-Acetamido-2-deoxy-D-gulopyranose, 2-Acetamido-2-deoxy-L-idopyranose, 2-Acetamido-2-deoxy-D-talopyranose, 2-Acetamido-2,6-dideoxy-D-galactopyranose, 2-Acetamido-2,6-dideoxy-L-galactopyranose, 2-Acetamido-2,6-dideoxy-L-mannopyranose, 2-Acetamido-2,6-dideoxy-D-glucopyranose, 2-Acetamido-2,6-dideoxy-L-altropyranose, 2-Acetamido-2,6-dideoxy-D-talopyranose, D-Glucopyranuronic acid, D-Galactopyranuronic acid, D-Mannopyranuronic acid, D-Allopyranuronic acid, L-Altropyranuronic acid, D-Glucopyranuronic acid, L-Glucopyranuronic acid, L-Idopyranuronic acid, D-Talopyranuronic acid, Sialic acid, 5-Amino-3,5-dideoxy-D-glycero-D-galacto-non-2-ulosonic acid, 5-Acetamido-3, 5-dideoxy-D-glycero-D-galacto-non-2-ulosonic acid, 5-Glycolylamido-3,5-dideoxy-D-glycero-D-galacto-non-2-ulosonic acid, Erythritol, Arabinitol, Xylitol, Ribitol, Glucitol, Galactitol, Mannitol, D-ribo-Hex-2-ulopyranose, D-arabino-Hex-2-ulofuranose (D-fructofuranose), D-arabino-Hex-2-ulopyranose, L-xylo-Hex-2-ulopyranose, D-lyxo-Hex-2-ulopyranose, D-threo-Pent-2-ulopyranose, D-altro-Hept-2-ulopyranose, 3-C-(Hydroxymethyl)-D-erythrofuranose, 2,4,6-Trideoxy-2,4-diamino-D-glucopyranose, 6-Deoxy-3-O-methyl-D-glucose, 3-O-Methyl-D-rhamnose, 2,6-Dideoxy-3-methyl-D-ribo-hexose, 2-Amino-3-O—[(R)-1-carboxyethyl]-2-deoxy-D-glucopyranose, 2-Acetamido-3-O—[(R)-carboxyethyl]-2-deoxy-D-glucopyranose, 2-Glycolylamido-3-O—[(R)-1-carboxyethyl]-2-deoxy-D-glucopyranose, 3-Deoxy-D-lyxo-hept-2-ulopyranosonic acid, 3-Deoxy-D-manno-oct-2-ulopyranosonic acid, 3-Deoxy-D-glycero-D-galacto-non-2-ulopyranosonic acid, 5,7-Diamino-3,5,7,9-tetradeoxy-L-glycero-L-manno-non-2-ulopyranosonic acid, 5,7-Diamino-3,5,7,9-tetradeoxy-L-glycero-L-altro-non-2-ulopyranosonic acid, 5,7-Diamino-3,5,7,9-tetradeoxy-D-glycero-D-galacto-non-2-ulopyranosonic acid, 5,7-Diamino-3,5,7,9-tetradeoxy-D-glycero-D-talo-non-2-ulopyranosonic acid, glucose, galactose, N-acetylglucosamine, glucosamine, mannose, xylose, N-acetylmannosamine, N-acetylneuraminic acid, N-glycolylneuraminic acid, a sialic acid, N-acetylgalactosamine, galactosamine, fucose, rhamnose, glucuronic acid, gluconic acid, fructose and polyols.

The term “mammalian milk oligosaccharide” as used herein refers to oligosaccharides found in mammalian milk, such as but not limited to 3-fucosyllactose, 2′-fucosyllactose, 6-fucosyllactose, 2′,3-difucosyllactose, 2′,2-difucosyllactose, 3,4-difucosyllactose, 6′-sialyllactose, 3′-sialyllactose, 3,6-disialyllactose, 6,6′-disialyllactose, 3,6-disialyllacto-N-tetraose, lactodifucotetraose, lacto-N-tetraose, lacto-N-neotetraose, lacto-N-fucopentaose II, lacto-N-fucopentaose I, lacto-N-fucopentaose III, sialyllacto-N-tetraose c, sialyllacto-N-tetraose b, sialyllacto-N-tetraose a, lacto-N-difucohexaose I, lacto-N-difucohexaose II, lacto-N-hexaose, lacto-N-neohexaose, para-lacto-N-hexaose, monofucosylmonosialyllacto-N-tetraose c, monofucosyl para-lacto-N-hexaose, monofucosyllacto-N-hexaose III, isomeric fucosylated lacto-N-hexaose III, isomeric fucosylated lacto-N-hexaose I, sialyllacto-N-hexaose, sialyllacto-N-neohexaose II, difucosyl-para-lacto-N-hexaose, difucosyllacto-N-hexaose, difucosyllacto-N-hexaose a, difucosyllacto-N-hexaose c, galactosylated chitosan.

A “sialylated oligosaccharide,” as used herein, refers to a charged sialic acid containing oligosaccharide, i.e., an oligosaccharide having a sialic acid residue. It has an acidic nature. Some examples are 3′-SL (3′-sialyllactose), 3′-sialyllactosamine, 6′-SL (6′-sialyllactose), 6′-sialyllactosamine, oligosaccharides comprising 6′-sialyllactose, SGG hexasaccharide (Neu5Aca-2,3Gal beta-1,3GalNac beta-1,3Gala-1,4Gal beta-1,4Gal), sialylated tetrasaccharide (Neu5Aca-2,3Gal beta-1,4GlcNac beta-14GlcNAc), pentasaccharide L STD (Neu5Aca-2,3Gal beta-1,4GlcNac beta-1,3Gal beta-1,4Glc), sialylated lacto-N-biose, sialylated lacto-N-triose, sialylated lacto-N-tetraose, sialyllacto-N-neotetraose, monosialyllacto-N-hexaose, disialyllacto-N-hexaose I, monosialyllacto-N-neohexaose I, monosialyllacto-N-neohexaose II, disialyllacto-N-neohexaose, disialyllacto-N-tetraose, disialyllacto-N-hexaose II, sialyllacto-N-tetraose a, disialyllacto-N-hexaose I, sialyllacto-N-tetraose b, 3′-sialyl-3-fucosyllactose, di sialomonofucosyllacto-N-neohexaose, monofucosylmonosialyllacto-N-octaose (sialyl Lea), sialyllacto-N-fucohexaose II, disialyllacto-N-fucopentaose II, monofucosyldisialyllacto-N-tetraose and oligosaccharides bearing one or several sialic acid residue(s), including but not limited to: oligosaccharide moieties of the gangliosides selected from GM3 (3′sialyllactose, Neu5Aca-2,3Gal β-4Glc) and oligosaccharides comprising the GM3 motif, GD3 Neu5Aca-2,8Neu5Aca-2,3Gal β-1,4Glc GT3 (Neu5Aca-2,8Neu5Aca-2,8Neu5Aca-2,3Gal β-1,4Glc); GM2 GalNAc β-1,4(Neu5Aca-2,3)Gal β-1,4Glc, GM1 Gal β-1,3GalNAc β-1,4(Neu5Aca-2,3)Gal β-1,4Glc, GD1a Neu5Aca-2,3Gal β-1,3GalNAc β-1,4(Neu5Aca-2,3)Gal β-1,4Glc GT1a Neu5Aca-2,8Neu5Aca-2,3Gal β-1,3GalNAc β-1,4(Neu5Aca-2,3)Gal β-1,4Glc GD2 GalNAc β-1,4(Neu5Aca-2,8Neu5Aca2,3)Gal β-1,4Glc GT2 GspalNAc β-1,4(Neu5Aca-2,8Neu5Aca-2,8Neu5Aca2,3)Gal β-1,4Glc GD1b, Gal β-1,3GalNAc β-1,4(Neu5Aca-2,8Neu5Aca2,3)Gal β-1,4Glc GT1b Neu5Aca-2,3Gal β-1,3GalNAc β-1,4(Neu5Aca-2,8Neu5Aca2,3)Gal β-1,4Glc GQ1b Neu5Aca-2,8Neu5Aca-2,3Gal β-1,3GalNAc β-1,4(Neu5Aca-2,8Neu5Aca2,3)Gal β-1,4Glc GT1c Gal β-1,3GalNAc β-1,4(Neu5Aca-2,8Neu5Aca-2,8Neu5Aca2,3)Gal β-1,4Glc GQ1c, Neu5Aca-2,3Gal β-1,3GalNAc β-1,4(Neu5Aca-2,8Neu5Aca-2,8Neu5Aca2,3)Gal β-1,4Glc GP1c Neu5Aca-2,8Neu5Aca-2,3Gal β-1,3GalNAc β-1,4(Neu5Aca-2,8Neu5Aca-2,8Neu5Aca2,3)Gal β-1,4Glc GD1a Neu5Aca-2,3Gal β-1,3(Neu5Aca-2,6)GalNAc β-1,4Gal β-1,4Glc Fucosyl-GM1 Fuca-1,2Gal β-1,3GalNAc β-1,4(Neu5Aca-2,3)Gal β-1,4Glc; all of which may be extended to the production of the corresponding gangliosides by reacting the above oligosaccharide moieties with ceramide or synthetizing the above oligosaccharides on a ceramide;

A “fucosylated oligosaccharide,” as used herein, is generally understood in the state of the art as an oligosaccharide that is carrying a fucose-residue. Examples comprise 2′-fucosyllactose, 3-fucosyllactose, difucosyllactose, lactodifucotetraose (LDFT), Lacto-N-fucopentaose I (LNF I), Lacto-N-fucopentaose II (LNF II),), Lacto-N-fucopentaose III (LNF III), lacto-N-fucopentaose V (LNF V), lacto-N-neofucopentaose I, lacto-N-difucohexaose I (LDFH I), lacto-N-difucohexaose II (LDFH II), Monofucosyllacto-N-hexaose III (MFLNH III), Difucosyllacto-N-hexaose (DFLNHa), difucosyl-lacto-N-neohexaose.

A “neutral oligosaccharide,” as used herein, is generally understood in the state of the art as an oligosaccharide that has no negative charge originating from a carboxylic acid group. Examples of such neutral oligosaccharide are 2′-fucosyllactose, 3-fucosyllactose, 2′,3-difucosyllactose, lacto-N-triose II, lacto-N-tetraose, lacto-N-neotetraose, lacto-N-fucopentaose I, lacto-N-neofucopentaose I, lacto-N-fucopentaose II, lacto-N-fucopentaose III, lacto-N-fucopentaose V, lacto-N-neofucopentaose V, lacto-N-difucohexaose I, lacto-N-difucohexaose II, 6′-galactosyllactose, 3′-galactosyllactose, lacto-N-hexaose, lacto-N-neohexaose, para-lacto-N-hexaose, para-lacto-N-neohexaose, difucosyl-lacto-N-hexaose and difucosyl-lacto-N-neohexaose.

The term “KDO-oligosaccharide” as used herein refers to KDO linked to a monosaccharide unit within an oligosaccharide or an acceptor as defined herein. Examples include, but are not limited to, KDO-lactose, KDO-LacNAc, KDO-N-Acetyl-D-lactosamine, KDO-LNB, KDO-Lacto-N-biose.

The term “precursor” as used herein refers to substances that are taken up or synthetized by the cell for the specific production of an oligosaccharide. In this sense a precursor can be an acceptor as defined herein, but can also be another substance, metabolite, which is first modified within the cell as part of the biochemical synthesis route of the oligosaccharide. Examples of such precursors comprise the acceptors as defined herein, and/or glucose, galactose, fructose, glycerol, sialic acid, fucose, mannose, maltose, sucrose, lactose glucose-1-phosphate, galactose-1-phosphate, UDP-glucose, UDP-galactose, glucose-6-phosphate, fructose phosphate, fructose-1,6-bisphosphate, glycerol-3-phosphate, dihydroxyacetone, glyceraldehyde-3-phosphate, dihydroxyacetone-phosphate, glucosamine-6-phosphate, glucosamine, N-acetyl-glucosamine-6-phosphate, N-acetyl-glucosamine, N-acetyl-mannosamine, N-acetylmannosamine-6-phosphate, UDP-N-acetylglucosamine, N-acetylglucosamine-1-phosphate, N-acetylneuraminic acid (sialic acid), N-acetyl-Neuraminic acid-9 phosphate, CMP-sialic acid, mannose-6-phosphate, mannose-1-phosphate, GDP-mannose, GDP-4-dehydro-6-deoxy-α-D-mannose, and/or GDP-fucose.

The term “acceptor” as used herein refers to oligosaccharides that can be modified by a glycosyltransferase. Examples of such acceptors are lactose, lacto-N-biose (LNB), lacto-N-triose, lacto-N-tetraose (LNT), lacto-N-neotetraose (LNnT), N-acetyl-lactosamine (LacNAc), lacto-N-pentaose (LNP), lacto-N-neopentaose, para lacto-N-pentaose, para lacto-N-neopentaose, lacto-N-novopentaose I, lacto-N-hexaose (LNH), lacto-N-neohexaose (LNnH), para lacto-N-neohexaose (pLNnH), para lacto-N-hexaose (pLNH), lacto-N-heptaose, lacto-N-neoheptaose, para lacto-N-neoheptaose, para lacto-N-heptaose, lacto-N-octaose (LNO), lacto-N-neooctaose, iso lacto-N-octaose, para lacto-N-octaose, iso lacto-N-neooctaose, novo lacto-N-neooctaose, para lacto-N-neooctaose, iso lacto-N-nonaose, novo lacto-N-nonaose, lacto-N-nonaose, lacto-N-decaose, iso lacto-N-decaose, novo lacto-N-decaose, lacto-N-neodecaose, galactosyllactose, a lactose extended with 1, 2, 3, 4, 5, or a multiple of N-acetyllactosamine units and/or 1, 2, 3, 4, 5, or a multiple of, Lacto-N-biose units, and oligosaccharide containing 1 or multiple N-acetyllactosamine units and/or 1 or multiple lacto-N-biose units or an intermediate or fucosylated or sialylated versions thereof.

The term “KDO” or “3-Deoxy-d-manno-oct-2-ulosonic acid” or “keto-deoxyoctulosonate” is an ulosonic acid of a 2-ketooctose formed in many bacteria as part of their lipo-oligosaccharide structure.

The term “purified” refers to material that is substantially or essentially free from components that interfere with the activity of the biological molecule. For cells, saccharides, nucleic acids, and polypeptides, the term “purified” refers to material that is substantially or essentially free from components that normally accompany the material as found in its native state. Typically, purified saccharides, oligosaccharides, proteins or nucleic acids of the disclosure are at least about 50%, 55%, 60%, 65%, 70%, 75%, 80% or 85% pure, usually at least about 90%, 91%, 92%, 93%, 94%, 95%, 96%, 97%, 98%, or 99% pure as measured by band intensity on a silver stained gel or other method for determining purity. Purity or homogeneity can be indicated by a number of means well known in the art, such as polyacrylamide gel electrophoresis of a protein or nucleic acid sample, followed by visualization upon staining. For certain purposes high resolution will be needed and HPLC or a similar means for purification utilized. For oligosaccharides, e.g., 3-sialyllactose, purity can be determined using methods such as but not limited to thin layer chromatography, gas chromatography, NMR, HPLC, capillary electrophoresis or mass spectroscopy.

The terms “identical” or percent “identity” or % “identity” in the context of two or more nucleic acid or polypeptide sequences, refer to two or more sequences or subsequences that are the same or have a specified percentage of amino acid residues or nucleotides that are the same, when compared and aligned for maximum correspondence, as measured using sequence comparison algorithms or by visual inspection. For sequence comparison, one sequence acts as a reference sequence, to which test sequences are compared. When using a sequence comparison algorithm, test and reference sequences are inputted into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Percent identity may be calculated globally over the full-length sequence of the reference sequence, resulting in a global percent identity score. Alternatively, percent identity may be calculated over a partial sequence of the reference sequence, resulting in a local percent identity score. Using the full-length of the reference sequence in a local sequence alignment results in a global percent identity score between the test and the reference sequence.

Percent identity can be determined using different algorithms like, for example, BLAST and PSI-BLAST (Altschul et al., 1990, J. Mol. Biol. 215:3, 403-410; Altschul et al., 1997, Nucleic Acids Res. 25: 17, 3389-402), the Clustal Omega method (Sievers et al., 2011, Mol. Syst. Biol. 7:539), the MatGAT method (Campanella et al., 2003, BMC Bioinformatics, 4:29) or EMBOSS Needle (https://galaxy-iuc.github.io/emboss-5.0-docs/needle.html).

The BLAST (Basic Local Alignment Search Tool) method of alignment is an algorithm provided by the National Center for Biotechnology Information (NCBI) to compare sequences using default parameters. The program compares nucleotide or protein sequences to sequence databases and calculates the statistical significance. PSI-BLAST (Position-Specific Iterative Basic Local Alignment Search Tool) derives a position-specific scoring matrix (PSSM) or profile from the multiple sequence alignment of sequences detected above a given score threshold using protein-protein BLAST (BLASTp). The BLAST method can be used for pairwise or multiple sequence alignments. Pairwise Sequence Alignment is used to identify regions of similarity that may indicate functional, structural and/or evolutionary relationships between two biological sequences (protein or nucleic acid). The web interface for BLAST is available at: https://blast.ncbi.nlm.nih.gov/Blast.cgi.

Clustal Omega (Clustal W) is a multiple sequence alignment program that uses seeded guide trees and HMM profile-profile techniques to generate alignments between three or more sequences. It produces biologically meaningful multiple sequence alignments of divergent sequences. The web interface for Clustal W is available at www.ebi.ac.uk/Tools/msa/clustalo/. Default parameters for multiple sequence alignments and calculation of percent identity of protein sequences using the Clustal W method are: enabling de-alignment of input sequences: FALSE; enabling mbed-like clustering guide-tree: TRUE; enabling mbed-like clustering iteration: TRUE; Number of (combined guide-tree/HMM) iterations: default(0); Max Guide Tree Iterations: default [−1]; Max HMM Iterations: default [−1]; order: aligned.

MatGAT (Matrix Global Alignment Tool) is a computer application that generates similarity/identity matrices for DNA or protein sequences without needing pre-alignment of the data. The program performs a series of pairwise alignments using the Myers and Miller global alignment algorithm, calculates similarity and identity, and then places the results in a distance matrix. The user may specify which type of alignment matrix (e.g., BLOSUM50, BLOSUM62, and PAM250) to employ with their protein sequence examination.

EMBOSS Needle (https://galaxy-iuc.github.io/emboss-5.0-docs/needle.html) uses the Needleman-Wunsch global alignment algorithm to find the optimal alignment (including gaps) of two sequences when considering their entire length. The optimal alignment is ensured by dynamic programming methods by exploring all possible alignments and choosing the best. The Needleman-Wunsch algorithm is a member of the class of algorithms that can calculate the best score and alignment in the order of mn steps, (where “n” and “m” are the lengths of the two sequences). The gap open penalty (default 10.0) is the score taken away when a gap is created. The default value assumes you are using the EBLOSUM62 matrix for protein sequences. The gap extension (default 0.5) penalty is added to the standard gap penalty for each base or residue in the gap. This is how long gaps are penalized.

For the purposes of this disclosure, percent identity is determined using MatGAT2.01 (Campanella et al., 2003, BMC Bioinformatics 4:29). The following default parameters for protein are employed: (1) Gap cost Existence: 12 and Extension: 2; (2) The Matrix employed was BLOSUM50.

### DETAILED DESCRIPTION

This disclosure provides a microbial host cell. In particular, this disclosure provides a microbial cell naturally synthesizing KDO genetically modified to produce an oligosaccharide. The KDO biosynthesis of the cell is knocked out or rendered less functional.

In a preferred embodiment, the cell comprises at least one glycosyltransferase with affinity for CMP-KDO. In a further preferred embodiment, the glycosyltransferase is overexpressed.

In a specific preferred embodiment, the cell according to the disclosure, is capable of synthesizing a nucleotide sugar selected from the group: GDP-fucose, GDP-mannose, GDP-rhamnose, CMP-N-acetylneuraminic acid, CMP-N-glycolylneuraminic acid, UDP-glucose, dTDP-glucose, UDP-galactose, UDP-N-acetylmannosamine, UDP-N-acetylglucosamine, UDP-N-acetylgalactosamine, UDP-glucuronic acid, UDP-xylose, UDP-arabinose, and/or UDP-galacturonic acid.

The cell according to the disclosure can comprise i) an overexpressed ATP-dependent translocator encoding gene; and/or ii) an overexpressed inner membrane protein encoding gene; and/or iii) an overexpressed lauroyl acyltransferase encoding gene. Preferably, the inner membrane protein is a transmembrane transporter. Alternatively or additionally, the cell can comprise a modified endogenous ATP-dependent translocator encoding gene; and/or a modified endogenous inner membrane protein encoding gene. Preferably, the inner membrane protein is a transmembrane transporter.

In a preferred embodiment, the overexpression of the ATP-dependent translocator, the inner membrane protein and/or lauroyl acyltransferase is an overexpression of the endogenous gene encoding the protein. Alternatively, the overexpression is obtained by introducing and expressing the protein(s). It has been shown that overexpression of any of the above proteins provides for a reduced or abolished KDO-biosynthesis. This reduced and/or abolished KDO-biosynthesis provides for production of oligosaccharide without at the same time producing KDO-by-products or KDO-side products, such as KDO-lactose, in general KDO-oligosaccharide or KDO containing lipopolysaccharides.

In another preferred embodiment, modification of the endogenous ATP-dependent translocator encoding gene and/or the endogenous inner membrane protein encoding gene is a point-mutation. It has been shown herein that also a point mutation in any of the genes encoding the endogenous ATP-dependent translocator or inner membrane protein provides for a reduced or abolished KDO-biosynthesis.

In preferred embodiments of this disclosure, the ATP-dependent translocator has 80% or more sequence identity to SEQ ID NO: 4 and has ATP-dependent translocator activity; the inner membrane protein has 80% or more sequence identity to SEQ ID NO: 21 and has transmembrane transporter activity and/or the lauroyl acyltransferase has 80% or more sequence identity to SEQ ID NO: 1 and has lauroyl acyltransferase activity.

The amino acid sequence of the polypeptide used herein can be a sequence chosen from SEQ ID NOS: 4, 21 or 1 of the attached sequence listing. The amino acid sequence of the polypeptide can also be an amino acid sequence that has 80% or more sequence identity, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9% sequence identity to the full length amino acid sequence of any one of SEQ NO 4, 21 or 1.

In another preferred embodiment, the cell comprises a KDO-independent lauroyl acyltransferase encoding gene and/or an ATP-binding cassette multidrug transporter encoding gene. Preferably, the KDO-independent lauroyl acyltransferase encoding gene and/or the ATP-binding cassette multidrug transporter is/are i) introduced and expressed or ii) overexpressed in the cell. This type of LpxL proteins is independent of KDO modified Lipid IVa, allowing full acylation of the lipidA structure when the KDO biosynthesis pathway is knocked out.

Preferably, the cell comprises expression of the gene encoding the KDO-independent lauroyl acyltransferase of SEQ ID NO: 2 or SEQ ID NO: 3, or a polypeptide having at least 80% sequence identity thereto and having KDO-independent lauroyl acyltransferase activity. The amino acid sequence of the polypeptide used herein can be a sequence chosen from SEQ ID NO: 2 or 3 of the attached sequence listing. The amino acid sequence can also be an amino acid sequence that has 80% or more sequence identity, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9% sequence identity to the full length amino acid sequence of any one of SEQ ID NO: 2 or 3.

In a preferred embodiment of this disclosure, the ATP-binding cassette multidrug transporter is LmrA from Lactococcus lactis, preferably comprising SEQ ID NO: 22. In another preferred embodiment, the cell comprises expression of a gene encoding a polypeptide having 80% or more sequence identity to SEQ ID NO: 22 and having transmembrane transporter activity. The amino acid sequence of the polypeptide can also be an amino acid sequence that has 80% or more sequence identity, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9% sequence identity to the full length amino acid sequence of SEQ ID NO: 22.

In a specific embodiment of the disclosure, the cell is an Escherichia coli wherein an msbA encoding gene is overexpressed and/or an yhjD encoding gene is overexpressed and/or a LpxL encoding gene is overexpressed and/or an endogenous msbA encoding gene is modified and/or an endogenous yhjD encoding gene is modified.

In a further preferred embodiment, the microbial cell naturally synthesizing KDO and genetically modified to produce an oligosaccharide, as described herein additionally or alternatively comprises at least one of the genes selected from the group of genes encoding for D-arabinose 5-phosphate isomerase, 3-deoxy-D-manno-octulosonate 8-phosphate synthase, 3-deoxy-D-manno-octulosonate 8-phosphate phosphatase, and/or 3-deoxy-manno-octulosonate cytidylyltransferase, which is rendered less functional or knocked out.

The microbial cell according to the disclosure is genetically modified to produce an oligosaccharide. Such oligosaccharide can be any oligosaccharide as described herein. Preferably, the cell produces a neutral, sialylated or fucosylated oligosaccharide. More preferably a mammalian milk oligosaccharide, even more preferably chosen from fucosylated milk oligosaccharides, neutral milk oligosaccharides and/or sialylated milk oligosaccharides.

In a preferred embodiment, the glycosyltransferase is a sialyltransferase. Preferably, the cell is then producing sialylated oligosaccharide, preferably sialylated mammalian milk oligosaccharide as described herein.

The cell according to the disclosure described herein can further comprise a deleted or inactivated endogenous beta-galactosidase gene. In case the cell is an E. coli cell, preferably the deleted or inactivated beta-galactosidase gene is E. coli lacZ gene.

The cell according to the disclosure described herein can further comprise a deleted, inactivated, or mutated galactoside-O-acyltransferase encoding gene. In case the cell is an E. coli cell, preferably the deleted, inactivated, or mutated galactoside-O-acyltransferase encoding gene is E. coli lacA gene.

The cell according to the disclosure described herein can further comprise a nucleic acid sequence encoding at least one of the following additional proteins: an exporter protein or a permease exporting the synthesized oligosaccharide from the microbial cell. Such exporter or permease will enable the export of the oligosaccharide out of the cell.

The cell according to the disclosure described herein can further be genetically modified to contain a nucleic acid sequence encoding a glycosidase for the specific degradation of interfering oligosaccharides, such as intermediates, side products or endogenous oligosaccharides generated by bacterial host cell, wherein the expression of glycosidase is under control of a regulatory sequence.

The cell according to the disclosure described herein can further be an isolated microbial cell according to any invention described herein.

This disclosure also provides for the use of a cell according to any one of embodiments described herein, for the production of oligosaccharides substantially free of KDO-lactose and/or KDO-oligosaccharide.

This disclosure provides for a method for the fermentative production of an oligosaccharide substantially free of KDO-oligosaccharide, the method comprising: providing a microbial cell according to any one of the embodiments described herein, cultivating the cells in favorable growing conditions and optionally separating or isolating the oligosaccharide from the culture.

In a specific embodiment, this disclosure provides for a method for producing a sialylated oligosaccharide by fermentation through a genetically modified microbial cell naturally synthesizing KDO, the method comprising the steps of:
a) Obtaining a microbial cell naturally synthesizing KDO and being capable to produce sialylated oligosaccharides and expressing a sialyltransferase with affinity for CMP-KDO, and wherein the KDO-biosynthesis route of the cell is knocked out or rendered less functional;
b) Culturing the host cell from step a) in favorable growing conditions, thus producing the sialylated oligosaccharide; and
c) Optionally, separating or isolating the sialylated oligosaccharide from the culture medium.

The culture medium used in the methods described herein preferably comprises precursor for the production of the oligosaccharide, preferably the culture medium comprises any one or more of lactose, galactose, N-acetylglucosamine, N-acetyl-D-lactosamine, Lacto-N-biose, sialic acid, N-acetylneuraminic acid, fucose. Alternatively or additionally, the cell produces the precursor internally; preferably, the cell produces any one or more of lactose, N-acetyl-D-lactosamine, Lacto-N-biose, sialic acid, N-acetylneuraminic acid, fucose.

According to a preferred embodiment of the disclosure described herein, the microbial cell produces a sialylated oligosaccharide, preferably chosen from the group comprising 3′-sialyllactose, 6′-sialyllactose, disialyl lacto-N-tetraose, sialylated lacto-N-triose, sialylated lacto-N-tetraose, sialylated lacto-N-neotetraose, 3-fucosyl-3′-sialyllactose, lacto-N-sialylpentaose, LSTa, LSTb, LSTc, LSTd. Alternatively or additionally, the cell produces a fucosylated and/or neutral oligosaccharide as described herein.

According to a preferred embodiment of the disclosure described herein, the microbial cell is a Gram-negative microbial cell, preferably, the cell is an Escherichia coli strain, preferably an Escherichia coli strain, which is a K12 strain, more preferably, the Escherichia coli K12 strain is Escherichia coli K12 substr. MG1655.

This disclosure, however, contemplates the use of any type of Gram-negative bacterial cell in the construction of microbial cells naturally synthesizing KDO, but wherein the KDO biosynthesis is knocked out or rendered less functional. Examples of Gram-negative bacteria useful in this disclosure include, but are not limited to of Escherichia spp., Shigella spp., Salmonella spp., Campylobacter spp., Neisseria spp., Haemophilus spp., Aeromonas spp., Francisella spp., Yersinia spp., Klebsiella spp., Bordetella spp., Legionella spp., Citrobacter spp., Chlamydia spp., Brucella spp., Pseudomonas spp., Helicobacter spp., Moraxella spp., Stenotrophomonas spp., Bdellovibrio spp., Acinetobacter spp., Enterobacter spp. and Vibrio spp. Even more preferably, the host cell is selected from Escherichia spp., Salmonella spp., and Pseudomonas spp. In preferred embodiments, Escherichia coli is used. Examples of Escherichia strains that can be used include, but are not limited to, Escherichia coli B, Escherichia coli C, Escherichia coli W, Escherichia coli K12, Escherichia coli Nissle. More specifically, the latter term relates to cultivated Escherichia coli strains—designated as E. coli K12 strains—which are well-adapted to the laboratory environment, and, unlike wild-type strains, have lost their ability to thrive in the intestine. Well-known examples of the E. coli K12 strains are K12 Wild type, W3110, MG1655, M182, MC1000, MC1060, MC1061, MC4100, JM101, NZN111 and AA200. Hence, this disclosure specifically relates to a mutated and/or transformed Escherichia coli strain as indicated above wherein the E. coli strain is a K12 strain. More specifically, this disclosure relates to a mutated and/or transformed Escherichia coli strain as indicated above wherein the K12 strain is E. coli K12 substr. MG1655.

Alternatively, the E. coli is selected from the group consisting of K-12 strain, W3110, MG1655, B/r, BL21, O157:h7, 042, 101-1,1180, 1357, 1412, 1520, 1827-70, 2362-75, 3431, 53638, 83972, 929-78, 98NK2, ABU 83972, B, B088, B171, B185, B354, B646, B7A, C, c7122, CFT073, DH1, DH5a, E110019, E128010, E74/68, E851/71, EAEC 042, EPECa11, EPECa12, EPECa14, ETEC, H10407, F11, F18+, FVEC1302, FVEC1412, GEMS EPEC1, HB101, HT115, K011, LF82, LT-41, LT-62, LT-68, MS107-1, MS119-7, MS124-1, MS 145-7, MS 79-2, MS 85-1, NCTC 86, Nissle 1917, NT:H19, NT:H40, NU14, O103:H2, O103:HNM, O103:K+, O104:H12, O108:H25, O109:H9, O111H−, O111:H19, O111:H2, O111:H21, O111:NM, O115:H−, O115:HMN, O115:K+, O119:H6, O119:UT, O124:H40, O127a:H6, O127:H6, O128:H2, O131:H25, O136:H−, O139:H28 (strain E24377A/ETEC), O13:H11, O142:H6, O145:H−, O153:H21, O153:H7, O154:H9, O157:12, O157:H−, O157:H12, O157:H43, O157:H45, O157:H7 EDL933, O157:NM, O15:NM, O177:H11, O17:K52:H18 (strain UMN026/ExPEC), O180:H−, O1:K1/APEC, O26, O26:H−, O26:H11, O26:H11:K60, O26:NM, O41:H−, O45:K1 (strain S88/ExPEC), O51:H−, O55:H51, O55:H6, O55:H7, O5:H−, O6, O63:H6, O63:HNM, O6:K15:H31 (strain 536/UPEC), O7:K1 (strain IAI39/ExPEC), O8 (strain IAI1), O81 (strain ED1a), O84:H−, O86a:H34, O86a:H40, O90:H8, O91:H21, O9:H4 (strain HS), O9:H51, ONT:H−, ONT:H25, OP50, Orough:H12, Orough:H19, Orough:H34, Orough:H37, Orough:H9, OUT:H12, OUT:H45, OUT:H6, OUT:H7, OUT:HNM, OUT:NM, RN587/1, RS218, 55989/EAEC, B/BL21, B/BL21-DE3, SE11, SMS-3-5/SECEC, UTI89/UPEC, TA004, TA155, TX1999, and Vir68.

In the methods of the disclosure, the oligosaccharide can be isolated from the culture medium by means of unit operation selected from the group comprising centrifugation, filtration, microfiltration, ultrafiltration, nanofiltration, ion exchange, electrodialysis, chromatography, simulated moving bed chromatography, simulated moving bed ion exchange, evaporation, precipitation, crystallization, spray drying and any combination thereof.

In a preferred embodiment of the methods of the disclosure, the produced oligosaccharide or mix of oligosaccharides is separated from the culture.

As used herein, the term “separating” means harvesting, collecting or retrieving the oligosaccharide from the host cell and/or the medium of its growth as explained herein.

Oligosaccharide can be separated in a conventional manner from the culture or aqueous culture medium, in which the mixture was made. In case the oligosaccharide is still present in the cells producing the oligosaccharide, conventional manners to free or to extract the oligosaccharide out of the cells can be used, such as cell destruction using high pH, heat shock, sonication, French press, homogenization, enzymatic hydrolysis, chemical hydrolysis, solvent hydrolysis, detergent, hydrolysis, . . . . The culture medium, reaction mixture and/or cell extract, together and separately called oligosaccharide containing mixture or culture, can then be further used for separating the oligosaccharide.

Typically oligosaccharides are purified by first removing macro components, i.e., first the cells and cell debris, then the smaller components, i.e., proteins, endotoxins and other components between 1000 Da and 1000 kDa and then the oligosaccharide is desalted by means of retaining the oligosaccharide with a nanofiltration membrane or with electrodialysis in a first step and ion exchange in a second step, which consists of a cation exchange resin and anion exchange resin, wherein most preferably the cation exchange chromatography is performed before the anion exchange chromatography. These steps do not separate sugars with a small difference in degree of polymerization from each other. The separation is done, for instance, by chromatographical separation.

Separation preferably involves clarifying the oligosaccharide containing mixtures to remove suspended particulates and contaminants, particularly cells, cell components, insoluble metabolites and debris produced by culturing the genetically modified cell and/or performing the enzymatic reaction. In this step, the oligosaccharide containing mixture can be clarified in a conventional manner. Preferably, the oligosaccharide containing mixture is clarified by centrifugation, flocculation, decantation and/or filtration. A second step of separating the oligosaccharide from the oligosaccharide containing mixture preferably involves removing substantially all the proteins, as well as peptides, amino acids, RNA and DNA and any endotoxins and glycolipids that could interfere with the subsequent separation step, from the oligosaccharide containing mixture, preferably after it has been clarified. In this step, proteins and related impurities can be removed from the oligosaccharide containing mixture in a conventional manner. Preferably, proteins, salts, by-products, color, and other related impurities are removed from the oligosaccharide containing mixture by ultrafiltration, nanofiltration, reverse osmosis, microfiltration, activated charcoal or carbon treatment, tangential flow high-performance filtration, tangential flow ultrafiltration, affinity chromatography, ion exchange chromatography (such as but not limited to cation exchange, anion exchange, mixed bed ion exchange), hydrophobic interaction chromatography and/or gel filtration (i.e., size exclusion chromatography), particularly by chromatography, more particularly by ion exchange chromatography or hydrophobic interaction chromatography or ligand exchange chromatography. With the exception of size exclusion chromatography, proteins and related impurities are retained by a chromatography medium or a selected membrane, while oligosaccharide remains in the oligosaccharide containing mixture.

Contaminating compounds with a molecular weight above 1000 Da (dalton) are removed by means of ultrafiltration membranes with a cut-off above 1000 Da to approximately 1000 kDa. The membrane retains the contaminant and the oligosaccharide goes to the filtrate. Typical ultrafiltration principles are well known in the art and are based on Tubular modules, Hollow fiber, spiral-wound or plates. These are used in cross flow conditions or as a dead-end filtration. The membrane composition is well known and available from several vendors, and is composed of PES (Polyethylene sulfone), polyvinylpyrrolidone, PAN (Polyacrylonitrile), PA (Poly-amide), Polyvinylidene difluoride (PVDF), NC (Nitrocellulose), ceramic materials or combinations thereof.

Components smaller than the oligosaccharide, for instance, monosaccharides, salts, disaccharides, acids, bases, medium constituents are separated by means of a nano-filtration or/and electrodialysis. Such membranes have molecular weight cut-offs between 100 Da and 1000 Da. For an oligosaccharide such as 3′-sialyllactose or 6′-sialyllactose the optimal cut-off is between 300 Da and 500 Da, minimizing losses in the filtrate. Typical membrane compositions are well known and are, for example, polyamide (PA), TFC, PA-TFC, Polypiperazine-amide, PES, Cellulose Acetate or combinations thereof.

The oligosaccharide is further isolated from the culture medium and/or cell with or without further purification steps by evaporation, lyophilization, crystallization, precipitation, and/or drying, spray drying. Further purification steps allow the formulation of oligosaccharide in combination with other oligosaccharides and/or products, for instance, but not limited to the co-formulation by means of spray drying, drying or lyophilization or concentration by means of evaporation in liquid form.

In an even further aspect, this disclosure also provides for a further purification of the oligosaccharide. A further purification of the oligosaccharide may be accomplished, for example, by use of (activated) charcoal or carbon, nanofiltration, ultrafiltration or ion exchange to remove any remaining DNA, protein, LPS, endotoxins, or other impurity. Alcohols, such as ethanol, and aqueous alcohol mixtures can also be used. Another purification step is accomplished by crystallization or precipitation of the product. Another purification step is to spray dry or lyophilize oligosaccharide.

The separated and preferably also purified oligosaccharide can be used as a supplement in infant formulas and for treating various diseases in new-born infants.

In a specific embodiment, an oligosaccharide is produced by the cell according to any one of embodiments described herein and/or according to the method described in any one of embodiments described herein. The oligosaccharide is added to food formulation, feed formulation, pharmaceutical formulation, cosmetic formulation, or agrochemical formulation.

Moreover, this disclosure relates to the following specific embodiments:

1. Microbial cell naturally synthesizing KDO genetically modified to produce an oligosaccharide, wherein the KDO biosynthesis of the cell is knocked out or rendered less functional.

2. A cell according to embodiment 1, wherein the cell comprises at least one glycosyltransferase with affinity for CMP-KDO, preferably the glycosyltransferase is overexpressed.

3. A cell according to any one of embodiments 1 and 2, wherein the cell is capable of synthesizing a nucleotide sugar selected from the group: GDP-fucose, GDP-mannose, GDP-rhamnose, CMP-N-acetylneuraminic acid, CMP-N-glycolylneuraminic acid, UDP-glucose, dTDP-glucose, UDP-galactose, UDP-N-acetylmannosamine, UDP-N-acetylglucosamine, UDP-N-acetylgalactosamine, UDP-glucuronic acid, UDP-xylose, UDP-arabinose, and/or UDP-galacturonic acid.

4. The cell according to any one of embodiments 1 to 3, wherein an ATP-dependent translocator encoding gene is overexpressed and/or an inner membrane protein encoding gene is overexpressed and/or an lauroyl acyltransferase encoding gene is overexpressed and/or an endogenous ATP-dependent translocator encoding gene is modified and/or an endogenous inner membrane protein encoding gene is modified, preferably, the inner membrane protein is a transmembrane transporter.

5. The cell according to any one of embodiments 1 to 4, wherein the cell comprises a KDO-independent lauroyl acyltransferase encoding gene and/or wherein the cell comprises an ATP-binding cassette multidrug transporter encoding gene.

6. Cell according to any one of embodiments 1 to 5, wherein the cell comprises expression of a gene encoding a KDO-independent lauroyl acyltransferase of SEQ ID NO: 2 or SEQ ID NO: 3, or a protein having at least 80% sequence identity thereto and having KDO-independent lauroyl acyltransferase activity.

7. Cell according to any one of embodiments 1 to 6, wherein the cell is an Escherichia coli and wherein an msbA encoding gene is overexpressed and/or an yhjD encoding gene is overexpressed and/or a LpxL encoding gene is overexpressed and/or an endogenous msbA encoding gene is modified and/or an endogenous yhjD encoding gene is modified.

8. Cell according to any one of embodiments 1 to 7, wherein the ATP-dependent translocator has 80% or more sequence identity to SEQ ID NO: 4 and has ATP-dependent translocator activity; the inner membrane protein has 80% or more sequence identity to SEQ ID NO: 21 and has transmembrane transporter activity and the lauroyl acyltransferase has 80% or more sequence identity to SEQ ID NO: 1 and has lauroyl acyltransferase activity.

9. Cell according to any one of embodiments 1 to 8, wherein the overexpression of the ATP-dependent translocator, the inner membrane protein and/or lauroyl acyltransferase is an overexpression of the endogenous gene encoding the protein or obtained by introducing and expressing the protein.

10. Cell according to any one of embodiments 1 to 9, wherein the modification of the endogenous ATP-dependent translocator encoding gene and/or the endogenous inner membrane protein encoding gene is a point-mutation.

11. Cell according to any one of embodiments 1 to 10, wherein the KDO-independent lauroyl acyltransferase encoding gene and/or wherein the ATP-binding cassette multidrug transporter is i) introduced and expressed or ii) overexpressed in the cell.

12. Cell according to any one of embodiments 1 to 11, wherein the cell comprises expression of the gene encoding the KDO-independent lauroyl acyltransferase of SEQ ID NO: 2 or SEQ ID NO: 3, or a protein having at least 80% sequence identity thereto and having KDO-independent lauroyl acyltransferase activity.

13. Cell according to any one of embodiments 1 to 12, wherein the cell comprises expression of the gene encoding the ATP-binding cassette multidrug transporter of SEQ ID NO: 22, or a protein having at least 80% sequence identity thereto and having transmembrane transporter activity.

14. The cell according to any one of embodiments 1 to 13, wherein at least one of the genes selected from the group of genes encoding for D-arabinose 5-phosphate isomerase, 3-deoxy-D-manno-octulosonate 8-phosphate synthase, 3-deoxy-D-manno-octulosonate 8-phosphate phosphatase, and/or 3-deoxy-manno-octulosonate cytidylyltransferase is rendered less functional or knocked out.

15. Cell according to any one of embodiments 1 to 14, wherein the cell produces a neutral, sialylated and/or fucosylated oligosaccharide.

16. Cell according to any one of embodiments 1 to 15, wherein the cell produces a mammalian milk oligosaccharide, preferably a neutral, sialylated and/or fucosylated milk oligosaccharide.

17. Cell according to any one of embodiments 1 to 16, wherein the glycosyltransferase is a sialyltransferase.

18. Cell according to any one of embodiments 1 to 17, wherein the cell comprises a deleted or inactivated endogenous beta-galactosidase gene.

19. Cell according to embodiment 18, wherein the deleted or inactivated beta-galactosidase gene comprises E. coli lacZ gene.

20. Cell according to any one of the preceding embodiments, wherein the microbial cell further comprises a deleted, inactivated, or mutated galactoside-O-acyltransferase (lacA) encoding gene.

21. Cell according to any one of the preceding embodiments, wherein the cell further comprises a nucleic acid sequence encoding at least one of the following additional proteins: an exporter protein or a permease exporting the synthesized oligosaccharide from the microbial cell.

22. Cell according to any one of the preceding embodiments, wherein the cell is further genetically modified to contain a nucleic acid sequence encoding a glycosidase for the specific degradation of interfering oligosaccharides, such as intermediates, side products or endogenous oligosaccharides generated by bacterial host cell, wherein the expression of the glycosidase is under control of a regulatory sequence.

23. An isolated microbial cell according to any one of embodiments 1 to 22.

24. Use of a cell according to any one of embodiments 1 to 23, for the production of sialylated oligosaccharides substantially free of KDO-lactose and/or KDO-oligosaccharide.

25. Method for the fermentative production of an oligosaccharide substantially free of KDO-oligosaccharide, the method comprising: providing a microbial cell according to any one of embodiments 1 to 23, cultivating the cells in favorable growing conditions and optionally separating or isolating the oligosaccharide from the culture.

26. Method for producing a sialylated oligosaccharide by fermentation through genetically modified microbial cell naturally synthesizing KDO, the method comprising the steps of:
a) Obtaining a microbial cell naturally synthesizing KDO and being capable to produce sialylated oligosaccharides and expressing a sialyltransferase with affinity for CMP-KDO, and wherein the KDO-biosynthesis route of the cell is knocked out or rendered less functional;
b) Culturing the cell from step a) in favorable growing conditions, thus producing the sialylated oligosaccharide and
c) Optionally, separating or isolating the sialylated oligosaccharide from the culture medium.

27. The method according to any one of embodiment 25 or 26, wherein the culture medium comprises precursor for the production of the oligosaccharide, preferably, the culture medium comprises any one or more of lactose, galactose, N-acetylglucosamine, N-acetyl-D-lactosamine, Lacto-N-biose, sialic acid, N-acetylneuraminic acid, fucose.

28. Method according to any one of embodiments 25 to 27, wherein the cell produces the precursor internally, preferably, the cell produces any one or more of lactose, N-acetyl-D-lactosamine, Lacto-N-biose, sialic acid, N-acetylneuraminic acid, fucose.

29. Method according to any one of embodiments 25 to 28, wherein the sialylated oligosaccharide is 3′-sialyllactose, 6′-sialyllactose, disialyl lacto-N-tetraose, sialylated lacto-N-triose, sialylated lacto-N-tetraose, sialylated lacto-N-neotetraose, 3-fucosyl-3′-sialyllactose, lacto-N-sialylpentaose LSTa, LSTb, LSTc, LSTd.

30. Microbial cell according to any one of embodiments 1 to 23, wherein the sialylated oligosaccharide is 3′-sialyllactose, 6′-sialyllactose, disialyl lacto-N-tetraose, sialylated lacto-N-triose, sialylated lacto-N-tetraose, sialylated lacto-N-neotetraose, 3-fucosyl-3′-sialyllactose, lacto-N-sialylpentaose LSTa, LSTb, LSTc, LSTd.

31. Method according to any one of embodiments 25 to 29, wherein the cell is a Gram-negative microbial cell, preferably, the cell is an Escherichia coli strain, preferably an Escherichia coli strain that is a K12 strain, more preferably the Escherichia coli K12 strain is Escherichia coli K12 substr. MG1655.

32. Microbial cell according to any one of claim 1 to 23, or 30, wherein the cell is a Gram-negative microbial cell, preferably, the cell is an Escherichia coli strain, preferably an Escherichia coli strain that is a K12 strain, more preferably the Escherichia coli K12 strain is Escherichia coli K12 substr. MG1655.

33. The method according to any one of embodiments 25 to 29, or 31, wherein the oligosaccharide is isolated from the culture medium by means of unit operation selected from the group comprising centrifugation, filtration, microfiltration, ultrafiltration, nanofiltration, ion exchange, electrodialysis, chromatography, simulated moving bed chromatography, simulated moving bed ion exchange, evaporation, precipitation, crystallization, spray drying and any combination thereof.

34. An oligosaccharide produced by the cell according to any one of embodiments 1 to 23, 30 or 32 and/or according to the method described in any one of embodiments 25 to 29, 31 or 33, wherein the oligosaccharide is added to food formulation, feed formulation, pharmaceutical formulation, cosmetic formulation, or agrochemical formulation.

The following examples will serve as further illustration and clarification of this disclosure and are not intended to be limiting.

### EXAMPLES

### Example 1: Material and Methods Escherichia coli

### Media

Three different media were used, namely a rich Luria Broth (LB), a minimal medium for shake flask (MMsf) and a minimal medium for fermentation (MMf). Both minimal media use a trace element mix.

Trace element mix consisted of 3.6 g/L FeCl₂.4H₂O, 5 g/L CaCl₂.2H₂O, 1.3 g/L MnCl₂.2H₂O, 0.38 g/L CuCl₂.2H₂O, 0.5 g/L CoCl₂.6H₂O, 0.94 g/L ZnCl₂, 0.0311 g/L H₃BO₄, 0.4 g/L Na₂EDTA.2H₂O and 1.01 g/L thiamine.HCl. The molybdate solution contained 0.967 g/L NaMoO₄.2H₂O. The selenium solution contained 42 g/L SeO₂.

The Luria Broth (LB) medium consisted of 1% tryptone peptone (Difco, Erembodegem, Belgium), 0.5% yeast extract (Difco) and 0.5% sodium chloride (VWR. Leuven, Belgium). Luria Broth agar (LBA) plates consisted of the LB media, with 12 g/L agar (Difco, Erembodegem, Belgium) added.

The minimal medium for the shake flasks (MMsf) experiments contained 2.00 g/L NH₄Cl, 5.00 g/L (NH₄)₂SO₄, 2.993 g/L KH₂PO₄, 7.315 g/L K₂HPO₄, 8.372 g/L MOPS, 0.5 g/L NaCl, 0.5 g/L MgSO₄.7H₂O, 14.26 g/L sucrose or another carbon source when specified in the examples, 1 ml/L trace element mix, 100 μl/L molybdate solution, and 1 mL/L selenium solution. The medium was set to a pH of 7 with 1 M KOH. Depending on the experiment lactose, LNB or LacNAc could be added as a precursor.

The minimal medium for fermentations (MMf) contained 6.75 g/L NH₄Cl, 1.25 g/L (NH₄)₂SO₄, 2.93 g/L KH₂PO₄ and 7.31 g/L KH₂PO₄, 0.5 g/L NaCl, 0.5 g/L MgSO₄.7H2O, 14.26 g/L sucrose, 1 mL/L trace element mix, 100 μL/L molybdate solution, and 1 mL/L selenium solution with the same composition as described above.

Complex medium, e.g., LB, was sterilized by autoclaving (121° C., 21′) and minimal medium by filtration (0.22 μm Sartorius). When necessary, the medium was made selective by adding an antibiotic (e.g., ampicillin (100 mg/L), chloramphenicol (20 mg/L), carbenicillin (100 mg/L), spectinomycin (40 mg/L) and/or kanamycin (50 mg/L)).

### Plasmids

pKD46 (Red helper plasmid, Ampicillin resistance), pKD3 (contains an FRT-flanked chloramphenicol resistance (cat) gene), pKD4 (contains an FRT-flanked kanamycin resistance (kan) gene), and pCP20 (expresses FLP recombinase activity) plasmids were obtained from Prof. R. Cunin (Vrije Universiteit Brussel, Belgium in 2007).

Plasmids for additional sialyltransferase expression were constructed in a pSC101 or a p15A ori containing backbone vector, respectively, using Golden Gate assembly. The sialyltransferases used in the enclosed examples are an alpha-2,6-sialyltransferases from Photobacterium sp. JT-ISH-224 and an alpha-2,3-sialyltransferase from Neisseria meningitidis with protein sequence SEQ ID 06 and 07, respectively. Table 1 gives an overview of the genes used to allow the deletion of the KDO biosynthetic pathway and to allow production of sialylated oligosaccharides. Genes that needed to be expressed, be it from a plasmid or from the genome were synthetically synthesized with one of the following companies: DNA2.0, Gen9, IDT or Twist Bioscience, or cloned in the genome of the original organism E. coli K12 and wherein specific genetic mutations are introduced via kits such as Quickchange site-directed mutagenesis (NEB). Expression could be further facilitated by optimizing the codon usage to the codon usage of the expression host. Genes were optimized using the tools of the supplier.

### Transcription Units

Heterologous genes allowing a deletion of the KDO biosynthetic pathway genes and sialyltransferase genes were expressed in different transcriptional units (TUs) using specific promoter, UTR and terminator combinations as enlisted in Table 2. The genes were expressed using promoters from Mutalik et al. (Nat. Methods 2013, No. 10, 354-360), as described herein as “PROM0005,” “PROM0006,” “PROM0012,” “PROM0035,” and “PROM0052” and a promoter from the Anderson promoter library (http://parts.igem.org/Promoters/Catalog/Anderson), as described herein as “PROM0024,” an IPTG-inducible promoter described herein as “PROM0017” and an inducible arabinose promotor as described herein as “PROM0018.” UTRs used as described herein as “UTR0002,” “UTR0006,” “UTR0010,” “UTR0017,” “UTR0018,” “UTR0029,” and “UTR0049” were obtained from Mutalik et al. (Nat. Methods 2013, No. 10, 354-360). Terminators used in the examples are described as “TER0002” (Orosz et al. (Eur. J. Biochem. 1991, 201, 653-59)) and as “TER0004” obtained from Kim et al. (Mol. Cells 1997 Feb. 28; 7(1):110-4). Table 2 shows the overview of the transcriptional units used in the examples by combination of the above promoters, UTRs and terminators.

TABLE 1

| SEQ ID | Name/ | | | Country of origin |
|---|---|---|---|---|
| NO: | TCDB | | | of digital sequence |
| (protein) | group | Organism | Origin | information |
| 01 | EcLpxL | E. coli K12 substr. MG1655 | Synthetic | USA |
| 02 | FtLpxL1 | Francisella tularensis subsp. | Synthetic | USA |
| | | Novicida | | |
| 03 | FtLpxL2 | Francisella tularensis subsp. | Synthetic | USA |
| | | Novicida | | |
| 04 | EcMsbA | E. coli K12 substr. MG1655 | Synthetic | USA |
| 05 | NmNeuA | N. meningitidis | Synthetic | UK |
| 06 | PbST224 | Photobacterium sp. JT-ISH-224 | Synthetic | Japan |
| 07 | NmST | N. meningitidis | Synthetic | UK |
| 08 | EclacY | Escherichia coli K12 | Synthetic | US |
| 09 | EwcscB | Escherichia coli W | Synthetic | US |
| 10 | BaSP | Bifidobacterium adolescentis | Synthetic | Germany |
| 11 | Zmfrk | Zymomonas mobilis | Synthetic | UK |
| 12 | EcglmS | Escherichia coli K12 | Synthetic | US |
| 13 | ScGNA1 | Saccharomyces cerevisiae | Synthetic | US |
| 14 | BoAGE | Bacteroides ovatus | Synthetic | US |
| 15 | CjneuB | Campylobacter jejuni | Synthetic | US |
| 16 | NmlgtA | Neisseria meningitidis | Synthetic | UK |
| 17 | NmlgtB | Neisseria meningitidis | Synthetic | UK |
| 18 | EcwbgO | E. coli O55:H7 | Synthetic | Germany |
| 19 | CjneuC | Campylobacter jejuni | Synthetic | US |
| 20 | kdsA | E. coli K12 substr. MG1655 | Synthetic | US |
| 21 | yhjD | E. coli K12 substr. MG1655 | Synthetic | US |
| 22 | LmrA | Lactococcus lactis | Synthetic | NA |

TABLE 2

| | Promoter | | | Terminator |
|---|---|---|---|---|
| TU number | part | CDS part | UTR part | part |
| TU 01 | PROM0012 | EcLpxL | UTR0049 | TER0004 |
| TU 02 | PROM0012 | EcLpxL | UTR0002 | TER0004 |
| TU 03 | PROM0012 | EcLpxL | UTR0017 | TER0004 |
| TU 04 | PROM0006 | EcLpxL | UTR0049 | TER0004 |
| TU 05 | PROM0006 | EcLpxL | UTR0002 | TER0004 |
| TU 06 | PROM0006 | EcLpxL | UTR0017 | TER0004 |
| TU 07 | PROM0052 | EcLpxL | UTR0029 | TER0004 |
| TU 08 | PROM0024 | EcLpxL | UTR0049 | TER0004 |
| TU 09 | PROM0024 | EcLpxL | UTR0002 | TER0004 |
| TU 10 | PROM0024 | EcLpxL | UTR0017 | TER0004 |
| TU 11 | PROM0017 | EcLpxL | UTR0006 | TER0004 |
| TU 12 | PROM0018 | EcLpxL | UTR0002 | TER0004 |
| TU 13 | PROM0012 | FtLpxL1 | UTR0049 | TER0004 |
| TU 14 | PROM0012 | FtLpxL1 | UTR0002 | TER0004 |
| TU 15 | PROM0012 | FtLpxL1 | UTR0017 | TER0004 |
| TU 16 | PROM0006 | FtLpxL1 | UTR0049 | TER0004 |
| TU 17 | PROM0006 | FtLpxL1 | UTR0002 | TER0004 |
| TU 18 | PROM0006 | FtLpxL1 | UTR0017 | TER0004 |
| TU 19 | PROM0052 | FtLpxL1 | UTR0029 | TER0004 |
| TU 20 | PROM0024 | FtLpxL1 | UTR0049 | TER0004 |
| TU 21 | PROM0024 | FtLpxL1 | UTR0002 | TER0004 |
| TU 22 | PROM0024 | FtLpxL1 | UTR0017 | TER0004 |
| TU 23 | PROM0012 | FtLpxL2 | UTR0049 | TER0004 |
| TU 24 | PROM0012 | FtLpxL2 | UTR0002 | TER0004 |
| TU 25 | PROM0012 | FtLpxL2 | UTR0017 | TER0004 |
| TU 26 | PROM0006 | FtLpxL2 | UTR0049 | TER0004 |
| TU 27 | PROM0006 | FtLpxL2 | UTR0002 | TER0004 |
| TU 28 | PROM0006 | FtLpxL2 | UTR0017 | TER0004 |
| TU 29 | PROM0052 | FtLpxL2 | UTR0029 | TER0004 |
| TU 30 | PROM0024 | FtLpxL2 | UTR0049 | TER0004 |
| TU 31 | PROM0024 | FtLpxL2 | UTR0002 | TER0004 |
| TU 32 | PROM0024 | FtLpxL2 | UTR0017 | TER0004 |
| TU 33 | PROM0017 | FtLpxL2 | UTR0006 | TER0004 |
| TU 34 | PROM0018 | FtLpxL2 | UTR0002 | TER0004 |
| TU 35 | PROM0035 | NmNeuA | UTR0018 | TER0002 |
| TU 36 | PROM0005 | PbST224 | UTR0010 | TER0002 |
| TU 37 | PROM0005 | NmST | UTR0010 | TER0002 |

### Strains and Mutations

Escherichia coli K12 substr. MG1655 [lambda⁻, F⁻, rph-1] was obtained from the Coli Genetic Stock Center (US), CGSC Strain #: 7740, in March 2007. Gene disruptions as well as gene introductions were performed using the technique published by Datsenko and Wanner (PNAS 97 (2000), 6640-6645). This technique is based on antibiotic selection after homologous recombination performed by lambda Red recombinase. Subsequent catalysis of a flippase recombinase ensures removal of the antibiotic selection cassette in the final production strain.

Transformants carrying a Red helper plasmid pKD46 were grown in 10 mL LB media with ampicillin, (100 mg/L) and L-arabinose (10 mM) at 30° C. to an OD^{600 nm} of 0.6. The cells were made electrocompetent by washing them with 50 mL of ice-cold water, a first time, and with 1 mL ice cold water, a second time. Then, the cells were resuspended in 50 μL of ice-cold water. Electroporation was done with 50 μL of cells and 10-100 ng of linear double-stranded-DNA product by using a Gene Pulser™ (BioRad) (600Ω, 25 μFD and 250 volts).

After electroporation, cells were added to 1 mL LB media incubated 1 hour at 37° C., and finally spread onto LB-agar containing 25 mg/L of chloramphenicol or 50 mg/L of kanamycin to select antibiotic resistant transformants. The selected mutants were verified by PCR with primers upstream and downstream of the modified region and were grown in LB-agar at 42° C. for the loss of the helper plasmid. The mutants were tested for ampicillin sensitivity.

The linear ds-DNA amplicons were obtained by PCR using pKD3, pKD4 and their derivates as template. The primers used had a part of the sequence complementary to the template and another part complementary to the side on the chromosomal DNA where the recombination must take place. For the genomic knockout, the region of homology was designed 50-nt upstream and 50-nt downstream of the start and stop codon of the gene of interest. For the genomic knock-in, the transcriptional starting point (+1) had to be respected. PCR products were PCR-purified, digested with Dpnl, repurified from an agarose gel, and suspended in elution buffer (5 mM Tris, pH 8.0).

The selected mutants (chloramphenicol or kanamycin resistant) were transformed with pCP20 plasmid, which is an ampicillin and chloramphenicol resistant plasmid that shows temperature-sensitive replication and thermal induction of FLP synthesis. The ampicillin-resistant transformants were selected at 30° C., after which a few were colony purified in LB at 42° C. and then tested for loss of all antibiotic resistance and of the FLP helper plasmid. The gene knockouts and knock-ins are checked with control primers (Fw/Rv-gene-out).

A sialic acid producing base strain derived from E. coli K12 substr. MG1655 was created by knocking out the genes asl, ldhA, poxB, atpI-gidB and ackA-pta, and knocking out the operons lacZYA, nagAB and the genes nanA, nanE and nanK. Additionally, the E. coli lacY gene (SEQ ID NO: 08) was introduced at the location of lacZYA. A fructose kinase gene (frk, SEQ ID NO: 11) originating from Zymomonas mobilis, an E. coli W sucrose transporter (cscB, SEQ ID NO: 09), a sucrose phosphorylase (SP, SEQ ID NO: 10) originating from Bifidobacterium adolescentis, an E. coli mutant fructose-6-P-aminotransferase (EcglmS*54, as described by Deng et al. (Biochimie 88, 419-29 (2006), SEQ ID NO: 12), glucosamine-6-P-aminotransferase from Saccharomyces cerevisiae (ScGNA1, SEQ ID NO: 13), an N-acetylglucosamine-2-epimerase from Bacteroides ovatus (BoAGE, SEQ ID NO: 14) and a sialic acid synthase from Campylobacter jejuni (CjneuB, SEQ ID NO: 15) were knocked in into the genome.

All strains are stored in cryovials at −80° C. (overnight LB culture mixed in a 1:1 ratio with 70% glycerol).

### Cultivation Conditions

A preculture of 96-well microtiter plate experiments was started from a cryovial, in 150 μL LB and was incubated overnight at 37° C. on an orbital shaker at 800 rpm. This culture was used as inoculum for a 96-well square microtiter plate, with 400 μL MMsf medium by diluting 400×. Each strain was grown in multiple wells of the 96-well plate as biological replicates. These final 96-well culture plates were then incubated at 37° C. on an orbital shaker at 800 rpm for 72 hours, or shorter, or longer.

Alternatively, a preculture for shake flask experiments was started from a cryovial, in 5 mL LB medium and was incubated for 8 hours at 37° C. on an orbital shaker at 200 rpm. From this preculture, 1 mL was transferred to 100 mL minimal medium (MMsf) in a 500 mL shake flask and incubated at 37° C. on an orbital shaker at 200 rpm for 72 hours, or shorter, or longer. This setup is used for shake flask experiments.

At the end of the cultivation experiment samples were taken from each well to measure the supernatant concentration (extracellular sugar concentrations, after 5 minutes spinning down the cells), or the whole broth concentration (by boiling the culture broth for 15 minutes at 60° C. before spinning down the cells (=intra- and extracellular sugar concentrations together).

Also, a dilution of the cultures was made to measure the optical density at 600 nm. The cell performance index or CPI is determined by dividing the oligosaccharide concentrations, e.g., sialyllactose concentrations, measured in the whole broth by the biomass, in relative percentages compared to the reference strain. The biomass is empirically determined to be approximately ⅓^{rd} of the optical density measured at 600 nm.

A preculture for the bioreactor was started from an entire 1 mL cryovial of a certain strain, inoculated in 250 mL or 500 mL of MMsf medium in a 1 L or 2.5 L shake flask and incubated for 24 hours at 37° C. on an orbital shaker at 200 rpm. A 5 L bioreactor was then inoculated (250 mL inoculum in 2 L batch medium); the process was controlled by MFCS control software (Sartorius Stedim Biotech, Melsungen, Germany). Culturing conditions were set to 37° C., and maximal stirring; pressure gas flow rates were dependent on the strain and bioreactor. The pH was controlled at 6.8 using 0.5 M H₂S0₄ and 20% NH₄OH. The exhaust gas was cooled. 10% solution of silicone antifoaming agent was added when foaming raised during the fermentation.

### Analytical Methods

### Optical Density

Cell density of the cultures was frequently monitored by measuring optical density at 600 nm (Implen Nanophotometer NP80, Westburg, Belgium or with a Spark 10M microplate reader, Tecan, Switzerland).

### Productivity

The specific productivity Qp is the specific production rate of the oligosaccharide product, typically expressed in mass units of product per mass unit of biomass per time unit (=g oligosaccharide/g biomass/h). The Qp value has been determined for each phase of the fermentation runs, i.e., Batch and Fed-Batch phase, by measuring both the amount of product and biomass formed at the end of each phase and the time frame each phase lasted.

The specific productivity Qs is the specific consumption rate of the substrate, e.g., sucrose, typically expressed in mass units of substrate per mass unit of biomass per time unit (=g sucrose/g biomass/h). The Qs value has been determined for each phase of the fermentation runs, i.e., Batch and Fed-Batch phase, by measuring both the total amount of sucrose consumed and biomass formed at the end of each phase and the time frame each phase lasted.

The yield on sucrose Ys is the fraction of product that is made from substrate and is typically expressed in mass unit of product per mass unit of substrate (=g oligosaccharide/g sucrose). The Ys has been determined for each phase of the fermentation runs, i.e., Batch and Fed-Batch phase, by measuring both the total amount of oligosaccharide produced and total amount of sucrose consumed at the end of each phase.

The yield on biomass Yx is the fraction of biomass that is made from substrate and is typically expressed in mass unit of biomass per mass unit of substrate (=g biomass/g sucrose). The Yp has been determined for each phase of the fermentation runs, i.e., Batch and Fed-Batch phase, by measuring both the total amount of biomass produced and total amount of sucrose consumed at the end of each phase.

The rate is the speed by which the product is made in a fermentation run, typically expressed in concentration of product made per time unit (=g oligosaccharide/L/h). The rate is determined by measuring the concentration of oligosaccharide that has been made at the end of the Fed-Batch phase and dividing this concentration by the total fermentation time.

The lactose conversion rate is the speed by which lactose is consumed in a fermentation run, typically expressed in mass units of lactose per time unit (=g lactose consumed/h). The lactose conversion rate is determined by measurement of the total lactose that is consumed during a fermentation run, divided by the total fermentation time. Similar conversion rates can be calculated for other precursors such as Lacto-N-biose, N-acetyl-lactosamine, Lacto-N-tetraose, or Lacto-N-neotetraose.

### Growth Rate/Speed Measurement

The maximal growth rate (μMax) was calculated based on the observed optical densities at 600 nm using the R package grofit.

### Liquid Chromatography

Standards for 6′-sialyllactose, 3′-sialyllactose, LNT and LNnT were synthetized in house. Other standards such as but not limited to lactose, sucrose, glucose, glycerol, fructose were purchased from Sigma, and LST, LacNAc and LNB were purchased from Carbosynth.

TLC analysis for oligosaccharide measurement was carried out on silica gels and the oligosaccharides were eluted with butanol-acetic acid-water (2:1:1 two runs). Sugars were detected by dipping the plate in orcinol sulfuric reagent and heating.

Carbohydrates were also analyzed via an UPLC-RI (Waters, USA) method, whereby RI (Refractive Index) detects the change in the refraction index of a mobile phase when containing a sample. The sugars were separated in an isocratic flow using an Acquity BEH Amide column (Waters, USA) and a mobile phase containing 70% acetonitrile, 26% ammonium acetate buffer and 4% methanol. The column size was 2.1×100 mm with 1.7 μm particle size. The temperature of the column was set at 25° C. and the pump flow rate was 0.13 mL/minute.

KDO-oligosaccharides and sialylated oligosaccharides were also measured by LC MS. Separation was performed on a Waters Acquity LC system with column heater set to 30° C. and a PGC column (Hypercarb 100×2.1 mm, 3 μm, Thermo Scientific). The injection volume was 5 μL. The mobile phases consisted of Milli-Q ultrapure water (A) and CH3CN (B), both containing 0.1% CH2O2 and delivered at a flow rate of 200 μL/minute. The gradient consisted of an initial increase from 0 to 12% B over 21 minutes, from 12 to 40% B over 11 minutes, from 40 to 100% B over 5 minutes. A washing step was conducted at 100% B for 5 minutes. The gradient was then decreased to 0% B over 1 minute and maintained at 0% B for 12 minutes for column equilibration. Total run time was 55 minutes. As mass-spectrometer, a Xevo TQ-MS was used with ESI in negative ionization mode, a cone voltage of 20V, desolvation temperature of 350° C. and nitrogen gas flow of 6501/hour. KDO (MW: 237 g/mol) containing oligosaccharides differ in mass from N-acetylneuraminate (MW: 309 g/mol) containing oligosaccharides and can be discriminated as such.

### Normalization of the Data

For all types of cultivation conditions, data obtained from the mutant strains was normalized against data obtained in identical cultivation conditions with reference strains having an identical genetic background as the mutant strains but having an active KDO biosynthesis pathway. The dashed horizontal line on each plot that is shown in the examples, indicates the setpoint to which all adaptations were normalized. All data is given in relative percentages to that setpoint.

### Example 2: Production Hosts for the Synthesis of an Oligosaccharide

A production host was created capable to synthesize a sialylated oligosaccharide. To do so, a sialic acid producing base strain as described in Example 1 was used for the overexpression on plasmid and/or on the genome of a CMP-sialic acid synthetase from Neisseria meningitidis (NmneuA, SEQ ID NO: 05, TU 35) and an α-2,6-sialyltransferase from Photobacterium sp. JT-ISH-224 (PbST224, SEQ ID NO: 06, TU 36). This transferase is known to accept both CMP-sialic acid and CMP-KDO. The production host was cultivated in lactose containing medium as described in Example 1 and formed KDO-lactose and 6′-SL. Oversialylation is avoided by adding lactose in excess to the medium, eliminating the formation of 6,6′-disialyllactose.

An alternative biosynthetic route toward sialylated oligosaccharides makes use of the internally available UDP-GlcNAc, by expressing a heterologous UDP-GlcNAc-2-epimerase (neuC), converting UDP-GlcNAc to N-acetylmannosamine, which is further converted into sialic acid by means of overexpression of a sialic acid synthase (NeuB) and to CMP-sialic acid by means of the overexpression of a CMP-sialic acid synthase (NeuA). To do so, a sialic acid producing base strain as described in Example 1 without a glucosamine-6-P-aminotransferase from Saccharomyces cerevisiae (ScGNA1, SEQ ID NO: 13) and without an N-acetylglucosamine epimerase from Bacteroides ovatus (BoAGE, SEQ ID NO: 14), but with an UDP-GlcNAc epimerase from Campylobacter jejuni (CjneuC, SEQ ID NO: 19) and a sialic acid synthase from Campylobacter jejuni (CjneuB, SEQ ID NO: 15) was created with the methods as described in Example 1. Further on, this sialic acid producing strain was used for the overexpression on plasmid and/or on the genome of a CMP-sialic acid synthetase from Neisseria meningitidis (NmneuA, SEQ ID NO: 05, TU 35) and an α-2,6-sialyltransferase from Photobacterium sp. JT-ISH-224 (PbST224, SEQ ID NO: 06, TU 36). This transferase is known to accept both CMP-sialic acid and CMP-KDO. The production host was cultivated in lactose containing medium as described in Example 1 and formed KDO-lactose and 6′-SL. Oversialylation is avoided by adding lactose in excess to the medium, eliminating the formation of 6,6′-disialyllactose.

Also, another production host was created as described in Example 1 capable to synthesize a sialylated oligosaccharide such as sialylated LacNAc (sLacNAc). To allow production of sLacNAc, a sialic acid base strain as described above is modified by the overexpression on plasmid and/or on the genome of a CMP-sialic acid synthetase from Neisseria meningitidis (NmneuA, SEQ ID NO: 05, TU 35) and an α-2,6-sialyltransferase from Photobacterium sp. JT-ISH-224 (PbST224, SEQ ID NO: 06, TU 36). This transferase is known to accept both CMP-sialic acid and CMP-KDO. The production host was cultivated in LacNAc containing medium as described in Example 1 and formed KDO-LacNAc and 6′-sLacNAc. Oversialylation is avoided by adding LacNAc in excess to the medium, eliminating the formation of 6,6′-disialylLacNAc.

Also, another production host was created as described in Example 1 capable to synthesize a sialylated oligosaccharide such as sialylated LNB (sLNB). To allow production of sLNB, a sialic acid base strain as described above is modified by the overexpression on plasmid and/or on the genome of a CMP-sialic acid synthetase from Neisseria meningitidis (NmneuA, SEQ ID NO: 05, TU 35) and an α-2,6-sialyltransferase from Photobacterium sp. JT-ISH-224 (PbST224, SEQ ID NO: 06, TU 36). This transferase is known to accept both CMP-sialic acid and CMP-KDO. The production host was cultivated in LNB containing medium as described in Example 1 and formed KDO-LNB and 6′-sLNB. Oversialylation is avoided by adding LNB in excess to the medium, eliminating the formation of 6,6′-disialylLNB.

### Example 3: A First Modified Production Host Wherein the KDO Biosynthesis Route is Knocked Out

A production host as described in Example 2 was further modified by introducing a point mutation in the endogenous msbA gene (SEQ ID NO: 04) at nucleotide 52 causing a C:G to T:A transition, changing the amino acid form a proline to a serine. The mutation allowed the deletion of the KDO biosynthesis pathway. In particular, the KDO biosynthesis pathway genes coding for D-arabinose 5-phosphate isomerase, which is for E. coli coded by gutQ and kdsD, the genes coding for 3-deoxy-D-manno-octulosonate 8-phosphate synthase, which is for E. coli coded by kdsA, the genes coding for 3-deoxy-D-manno-octulosonate 8-phosphate phosphatase, which is for E. coli coded by kdsC, and/or the gene coding for 3-deoxy-manno-octulosonate cytidylyltransferase, which is for E. coli coded by kdsB were deleted. Primers to construct these genetic deletions and the specific msbA mutation are listed in Table 3. Primers to check the specific KDO pathway gene knockouts are listed in Table 4.

The modified organism was cultivated as described in Example 1 and the formation of KDO-containing oligosaccharides was evaluated. No KDO-oligosaccharides were formed in this host, only 6′-SL, 6′-sLacNAc or 6′-sLNB depending on the used medium.

TABLE 3

| | Knockout (KO) / |
|---|---|
| | mutation |
| Primer sequence (5′ - 3′) | construction |
| attgtgctgcattaattaatcgacattttactcaagattaaggcgatcctccgcggtgcgggtgccaggg | kdsA KO |
| (SEQ ID NO: 23) | |
| Atgaaaaaagtcttaacgcagaacgctaatactttatttttcaagcaaaaaagactacgcccccaactgag | kdsA KO |
| agaac | |
| (SEQ ID NO: 24) | |
| tgctggaaacggaagcccgcgtgctgactgctgatgagagtaaatcatgaccgcggtgcgggtgccag | kdsB KO |
| (SEQ ID NO: 25) | |
| taacggtacgacactcctcccaaaattggctgaagtgtcgtgaagtgaaactacgcccccaactgagaga | kdsB KO |
| ac | |
| (SEQ ID NO: 26) | |
| catgatttactgcgtgcaggcgtagtgtaaagattcaaggataaacaacaccgcggtgcgggtgccagg | kdsC KO |
| g | |
| (SEQ ID NO: 27) | |
| agaaccgccagtgatagcacaatgataacccaacgtctggctttactcatctacgcccccaactgagaga | kdsC KO |
| ac | |
| (SEQ ID NO: 28) | |
| gttgtactggttatcgccaatactcgttgaataactggaaacgcattatgccgcggtgcgggtgccaggg | kdsP KO |
| (SEQ ID NO: 29) | |
| tttgctcattgttgtttatccttgaatctttacactacgcctgcacgcagctacgcccccaactgagagaac | kdsD KO |
| (SEQ ID NO:30) | |
| agagagcaatgagtgaagcactactgaacgcgggacgtcagacgttaatgccgcggtgcgggtgcca | gutQ KO |
| g | |
| (SEQ ID NO: 31) | |
| cggctggcgaaacgtctgggattgaaggattaaataatcccggcctgatactacgcccccaactgagag | gutQ KO |
| (SEQ ID NO: 32) | |
| gcatcgtctcaatctggtctcaaatgcataacgacaaagatctctctacgtggcagacattccgccgactgt | msbA nt 52: C:G |
| ggtcaacc | to T:A |
| (SEQ ID NO: 33) | |
| tgccgtctcactaaggtctcacagcttattggccaaactgcattttgtg | msbA nt 52: C:G |
| (SEQ ID NO: 34) | to T:A |
| gcatcgtctcaatctggtctcaaatgcataacgacaaagatctctctacg | msbA nt 148: |
| (SEQ ID NO: 35) | C:G to T:A |
| tatgcgtctctgtgacttaaggagcgataacatg | msbA nt 148: |
| (SEQ ID NO: 36) | C:G to T:A |
| actacgtctcatcacttcttgatgatggctttgg | msbA nt 148: |
| (SEQ ID NO: 37) | C:G to T:A |
| tgccgtctcactaaggtctcacagcttattggccaaactgcattttgtg | msbA nt 148: |
| (SEQ ID NO: 38) | C:G to T:A |
| gcatcgtctcaatctggtctcaaatgacgcaggaaaacgagatc | vhjD nt 400 C:G |
| (SEQ ID NO: 39) | to T:A |
| tatgcgtctcttacactgctgaacggcggtgttg | yhjD nt 400 C:G |
| (SEQ ID NO: 40) | to T:A |
| actacgtctcatgtacgactgtagggcttgtcg | yhjD nt 400 C:G |
| (SEQ ID NO: 41) | to T:A |
| atgccgtctcactaaggtctcacagcttaaggctgcgttttcccc | yhjD nt 400 C:G |
| (SEQ ID NO: 42) | to T:A |

TABLE 4

| | Knockout |
|---|---|
| Primer sequence (5′ - 3′) | (KO) check |
| tccggatggcgaaatttggc (SEQ ID NO: 43) | kdsA KO |
| tttactggcttcctggtggc (SEQ ID NO: 44) | kdsA KO |
| agatccctatgaaattcgcg (SEQ ID NO: 45) | kdsA KO |
| cgcatctggcactgtttgcc (SEQ ID NO: 46) | kdsA KO |
| ggtgacattaggatgctgcc (SEQ ID NO: 47) | kdsB KO |
| tctttgagttgttcccagcg (SEQ ID NO: 48) | kdsB KO |
| cgtctgcttgaaatcattgcc (SEQ ID NO: 49) | kdsB KO |
| aatggcttaattcggcacgc (SEQ ID NO: 50) | kdsB KO |
| atacgggcgatgagatcccg (SEQ ID NO: 51) | kdsC KO |
| ttctgggttatagacgagcg (SEQ ID NO: 52) | kdsC KO |
| tgaggcactgaacttaatgc (SEQ ID NO: 53) | kdsC KO |
| tgcgaagttgagagtctggc (SEQ ID NO: 54) | kdsC KO |
| gtgagtgatccccaatgtggc (SEQ ID NO: 55) | kdsD KO |
| cccgcgctgataacgccagg (SEQ ID NO: 56) | kdsD KO |
| ctgattgcagaagctgccg (SEQ ID NO: 57) | gutQ KO |
| gaagtggcggcggtgcccg (SEQ ID NO: 58) | gutQ KO |

### Example 4: A Second Modified Production Host Wherein the KDO Biosynthesis Route is Knocked Out

A production host as described in Example 2 was further modified by introducing a point mutation in the endogenous msbA gene (SEQ ID NO: 04) at base 148 causing a C:G to T:A transition, changing the amino acid form a proline to a serine. The mutation allowed the deletion of the KDO biosynthesis pathway. In particular, the KDO biosynthesis pathway genes coding for D-arabinose 5-phosphate isomerase, which is for E. coli coded by gutQ and kdsD, the genes coding for 3-deoxy-D-manno-octulosonate 8-phosphate synthase, which is for E. coli coded by kdsA, the genes coding for 3-deoxy-D-manno-octulosonate 8-phosphate phosphatase, which is for E. coli coded by kdsC, and/or the gene coding for 3-deoxy-manno-octulosonate cytidylyltransferase, which is for E. coli coded by kdsB were deleted. Primers to construct these genetic deletions and the specific msbA mutation are listed in Table 3. Primers to check the specific KDO pathway gene knockouts are listed in Table 4.

The modified organism was cultivated as described in Example 1 and the formation of KDO-containing oligosaccharides was evaluated. No KDO-oligosaccharides were formed in this host, only 6′-SL, 6′-sLacNAc or 6′-sLNB depending on the used medium.

### Example 5: A Third Modified Production Host Wherein the KDO Biosynthesis Route is Knocked Out

A production host as described in Example 2 was further modified by overexpressing the endogenous msbA (SEQ ID NO: 04) in the production host. The overexpression allowed the deletion of the KDO biosynthesis pathway. In particular, the KDO biosynthesis pathway genes coding for D-arabinose 5-phosphate isomerase, which is for E. coli coded by gutQ and kdsD, the genes coding for 3-deoxy-D-manno-octulosonate 8-phosphate synthase, which is for E. coli coded by kdsA, the genes coding for 3-deoxy-D-manno-octulosonate 8-phosphate phosphatase, which is for E. coli coded by kdsC, and/or the gene coding for 3-deoxy-manno-octulosonate cytidylyltransferase, which is for E. coli coded by kdsB were deleted. Primers to construct these genetic deletions and the specific msbA mutation are listed in Table 3. Primers to check the specific KDO pathway gene knockouts are listed in Table 4.

The modified organism was cultivated as described in Example 1 and the formation of KDO-containing oligosaccharides was evaluated. No KDO-oligosaccharides were formed in this host, only 6′-SL, 6′-sLacNAc or 6′-sLNB depending on the used medium.

### Example 6: A Fourth Modified Production Host Wherein the KDO Biosynthesis Route is Knocked Out

A production host as described in Example 2 was further modified by introducing a point mutation in the endogenous yhjD gene causing a C:G to T:A transition at nucleotide number 400 leading to a change from arginine to cysteine at amino acid position 134. The mutation allowed the deletion of the KDO biosynthesis pathway. In particular, the KDO biosynthesis pathway genes coding for D-arabinose 5-phosphate isomerase, which is for E. coli coded by gutQ and kdsD, the genes coding for 3-deoxy-D-manno-octulosonate 8-phosphate synthase, which is for E. coli coded by kdsA, the genes coding for 3-deoxy-D-manno-octulosonate 8-phosphate phosphatase, which is for E. coli coded by kdsC, and/or the gene coding for 3-deoxy-manno-octulosonate cytidylyltransferase, which is for E. coli coded by kdsB were deleted. Primers to construct these genetic deletions and the specific msbA mutation are listed in Table 3. Primers to check the specific KDO pathway gene knockouts are listed in Table 4.

The modified organism was cultivated as described in Example 1 and the formation of KDO-containing oligosaccharides was evaluated. No KDO-oligosaccharides were formed in this host, only 6′-SL, 6′-sLacNAc or 6′-sLNB depending on the used medium.

### Example 7: A Fifth Modified Production Host Wherein the KDO Biosynthesis Route is Knocked Out

A production host as described in Example 2 was further modified by introducing additional copies of the endogenous LpxL (SEQ ID NO: 01). The lpxL gene was overexpressed and evaluated in different transcription units (TU 01-TU 12). This overexpressed LpxL gene allowed the deletion of the KDO biosynthesis pathway. In particular, the KDO biosynthesis pathway genes coding for D-arabinose 5-phosphate isomerase, which is for E. coli coded by gutQ and kdsD, the genes coding for 3-deoxy-D-manno-octulosonate 8-phosphate synthase, which is for E. coli coded by kdsA, the genes coding for 3-deoxy-D-manno-octulosonate 8-phosphate phosphatase, which is for E. coli coded by kdsC, and/or the gene coding for 3-deoxy-manno-octulosonate cytidylyltransferase, which is for E. coli coded by kdsB were deleted. Primers to construct these genetic deletions and the specific msbA mutation are listed in Table 3. Primers to check the specific KDO pathway gene knockouts are listed in Table 4.

The modified organism was cultivated as described in Example 1 and the formation of KDO-containing oligosaccharides was evaluated. No KDO-oligosaccharides were formed in this host, only 6′-SL, 6′-sLacNAc or 6′-sLNB depending on the used medium.

### Example 8: A Sixth Modified Production Host Wherein the KDO Biosynthesis Route is Knocked Out

A production host as described in Example 2 was further modified by introducing an alternative LpxL gene originating from Francisella tularensis subsp. novicida, LpxL1 (SEQ ID NO: 02). This group of LpxL proteins is independent of KDO modified Lipid IVa, allowing full acylation of the lipidA structure when the KDO biosynthesis pathway is knocked out. The gene was overexpressed and evaluated in different transcription units (TU 13-TU 22). This overexpressed FtLpxL1 gene allowed the deletion of the KDO biosynthesis pathway. In particular, the KDO biosynthesis pathway genes coding for D-arabinose 5-phosphate isomerase, which is for E. coli coded by gutQ and kdsD, the genes coding for 3-deoxy-D-manno-octulosonate 8-phosphate synthase, which is for E. coli coded by kdsA, the genes coding for 3-deoxy-D-manno-octulosonate 8-phosphate phosphatase, which is for E. coli coded by kdsC, and/or the gene coding for 3-deoxy-manno-octulosonate cytidylyltransferase, which is for E. coli coded by kdsB were deleted. Primers to construct these genetic deletions and the specific msbA mutation are listed in Table 3. Primers to check the specific KDO pathway gene knockouts are listed in Table 4.

The modified organism was cultivated as described in Example 1 and the formation of KDO-containing oligosaccharides was evaluated. No KDO-oligosaccharides were formed in this host, only 6′-SL, 6′-sLacNAc or 6′-sLNB depending on the used medium.

### Example 9: A Seventh Modified Production Host Wherein the KDO Biosynthesis Route is Knocked Out

A production host as described in Example 2 was further modified by introducing an alternative LpxL gene originating from Francisella tularensis subsp. novicida, LpxL2 (SEQ ID NO: 03). This group of LpxL proteins is independent of KDO modified Lipid IVa, allowing full acylation of the lipidA structure when the KDO biosynthesis pathway is knocked out. The gene was overexpressed and evaluated in different transcription units (TU 23-34). This overexpressed FtLpxL2 gene allowed the deletion of the KDO biosynthesis pathway. In particular, the KDO biosynthesis pathway genes coding for D-arabinose 5-phosphate isomerase, which is for E. coli coded by gutQ and kdsD, the genes coding for 3-deoxy-D-manno-octulosonate 8-phosphate synthase, which is for E. coli coded by kdsA, the genes coding for 3-deoxy-D-manno-octulosonate 8-phosphate phosphatase, which is for E. coli coded by kdsC, and/or the gene coding for 3-deoxy-manno-octulosonate cytidylyltransferase, which is for E. coli coded by kdsB were deleted. Primers to construct these genetic deletions and the specific msbA mutation are listed in Table 3. Primers to check the specific KDO pathway gene knockouts are listed in Table 4.

The modified organism was cultivated as described in Example 1 and the formation of KDO-containing oligosaccharides was evaluated. No KDO-oligosaccharides were formed in this host, only 6′-SL, 6′-sLacNAc or 6′-sLNB depending on the used medium.

### Example 10: A Production Host for the Synthesis of LSTc

A production host as described in Example 2 wherein the genes encoding for a beta-1,3-GlcNAc transferase from Neisseria meningitidis (NmlgtA, SEQ ID NO: 16) and a beta-1,4-galactosyltransferase from Neisseria meningitidis (NmlgtB, SEQ ID NO: 17) are introduced in the genome, allowing the synthesis of the precursor LNnT.

This organism produces in the culture conditions supplemented with lactose as described in Example 1 sialylated lacto-N-neotetraose (LSTc, a-NeuNAc-(2-6)-b-Gal-(1-4)-b-GlcNAc-(1-3)-b-Gal-(1-4)-Glc), and KDO containing oligosaccharides such as KDO-lactose.

### Example 11: An Eighth Modified Production Host Wherein the KDO Biosynthesis Route is Knocked Out

The production host as described in Example 10 was further modified by introducing additional copies of EcLpxL (SEQ ID NO: 01), FtLpxL1 (SEQ ID NO: 02) or FtLpxL2 (SEQ ID NO: 03). The LpxL genes were overexpressed and evaluated in different transcription units (TU 01-TU 34). These gene overexpressions allowed the deletion of the KDO biosynthesis pathway. In particular, the KDO biosynthesis pathway genes coding for D-arabinose 5-phosphate isomerase, which is for E. coli coded by gutQ and kdsD, the genes coding for 3-deoxy-D-manno-octulosonate 8-phosphate synthase, which is for E. coli coded by kdsA, the genes coding for 3-deoxy-D-manno-octulosonate 8-phosphate phosphatase, which is for E. coli coded by kdsC, and/or the gene coding for 3-deoxy-manno-octulosonate cytidylyltransferase, which is for E. coli coded by kdsB were deleted. Primers to construct these genetic deletions and the specific msbA mutation are listed in Table 3. Primers to check the specific KDO pathway gene knockouts are listed in Table 4.

This organism was cultured in the culture conditions supplemented with lactose as described in Example 1 and produced the sialylated lacto-N-neotetraose (LSTc, a-NeuNAc-(2-6)-b-Gal-(1-4)-b-GlcNAc-(1-3)-b-Gal-(1-4)-Glc). No KDO-containing oligosaccharides were found in the culture medium.

### Example 12: A Ninth Modified Production Host Wherein the KDO Biosynthesis Route is Knocked Out

The production host as described in Example 10 was further modified by overexpressing the endogenous msbA (SEQ ID NO: 04) in the production host. The overexpression allowed the deletion of the KDO biosynthesis pathway. In particular, the KDO biosynthesis pathway genes coding for D-arabinose 5-phosphate isomerase, which is for E. coli coded by gutQ and kdsD, the genes coding for 3-deoxy-D-manno-octulosonate 8-phosphate synthase, which is for E. coli coded by kdsA, the genes coding for 3-deoxy-D-manno-octulosonate 8-phosphate phosphatase, which is for E. coli coded by kdsC, and/or the gene coding for 3-deoxy-manno-octulosonate cytidylyltransferase, which is for E. coli coded by kdsB were deleted. Primers to construct these genetic deletions and the specific msbA mutation are listed in Table 3. Primers to check the specific KDO pathway gene knockouts are listed in Table 4.

This organism was cultured in the culture conditions supplemented with lactose as described in Example 1 and produced the sialylated lacto-N-neotetraose (LSTc, a-NeuNAc-(2-6)-b-Gal-(1-4)-b-GlcNAc-(1-3)-b-Gal-(1-4)-Glc). No KDO-containing oligosaccharides were found in the culture medium.

### Example 13: A Production Host for the Synthesis of LSTb

The production host as described in Example 2 wherein the genes encoding for a beta-1,3-GlcNAc transferase from Neisseria meningitidis (NmlgtA, SEQ ID NO: 16) and a beta-1,3-galactosyltransferase from E. coli 055:H7 (EcwbgO, SEQ ID NO: 18) are introduced in the genome, allowing the synthesis of the precursor LNT. The organism is further modified with the α-2,6-sialyltransferase ST6gall or ST6Galll instead of the α-2,6-sialyltransferase from Photobacterium sp. JT-ISH-224.

This organism produces in the culture conditions supplemented with lactose as described in Example 1 sialylated lacto-N-tetraose (LSTb, a-NeuNAc-(2-6)-(b-D-Gal-[1-3])-b-D-GlcNAc-(1-3)-b-D-Gal-(1-4)-Glc), and KDO containing oligosaccharides such as KDO-lactose.

### Example 14: A Tenth Modified Production Host Wherein the KDO Biosynthesis Route is Knocked Out

The production host as described in Example 13 was further modified by introducing additional copies of EcLpxL (SEQ ID NO: 01), FtLpxL1 (SEQ ID NO: 02) or FtLpxL2 (SEQ ID NO: 03). The LpxL genes were overexpressed and evaluated in different transcription units (TU 01-TU 34). These gene overexpressions allowed the deletion of the KDO biosynthesis pathway. In particular, the KDO biosynthesis pathway genes coding for D-arabinose 5-phosphate isomerase, which is for E. coli coded by gutQ and kdsD, the genes coding for 3-deoxy-D-manno-octulosonate 8-phosphate synthase, which is for E. coli coded by kdsA, the genes coding for 3-deoxy-D-manno-octulosonate 8-phosphate phosphatase, which is for E. coli coded by kdsC, and/or the gene coding for 3-deoxy-manno-octulosonate cytidylyltransferase, which is for E. coli coded by kdsB were deleted. Primers to construct these genetic deletions and the specific msbA mutation are listed in Table 3. Primers to check the specific KDO pathway gene knockouts are listed in Table 4.

This organism was cultured in the culture conditions supplemented with lactose as described in Example 1 and produced sialylated lacto-N-tetraose (LSTb, a-NeuNAc-(2-6)-(b-D-Gal-[1-3])-b-D-GlcNAc-(1-3)-b-D-Gal-(1-4)-Glc). No KDO-containing oligosaccharides were found in the culture medium.

### Example 15: Modified Production Hosts Wherein the KDO Biosynthesis Route is Knocked Out for the Synthesis of 3′-Sialyllactose (3′-SL)

The production hosts as described in Example 3 to 9 were modified so that the 6′-SL forming transferase is replaced by a 3′-SL forming transferase. The α-2,3-sialyltransferase from Neisseria meningitidis (NmST, SEQ ID NO: 07) was expressed on a plasmid and/or on the genome within TU 37.

These organisms were cultured in the culture conditions supplemented with lactose, LacNAc or LNB as described in Example 1 and produced 3′-SL, 3′-sLacNAc or 3′-sLNB depending on the used medium. Within the culture fluid no KDO-containing oligosaccharides were found.

### Example 16: Modified Production Hosts Wherein the KDO Biosynthesis Route is Knocked Out for the Synthesis of LSTd

The production hosts as described in Example 15, wherein the genes encoding for a beta-1,3-GlcNAc transferase from Neisseria meningitidis (NmlgtA, SEQ ID NO: 16) and a beta-1,4-galactosyltransferase from Neisseria meningitidis (NmlgtB, SEQ ID NO: 17) are introduced in the genome, allowing the synthesis of the precursor LNnT.

These organisms were cultured in the culture conditions supplemented with lactose as described in Example 1 and produced LSTd (a-NeuNAc-(2-3)-b-D-Gal-(1-4)-b-D-GlcNAc-(1-3)-b-D-Gal-(1-4)-Glc). Within the culture fluid no KDO-containing oligosaccharides were found.

### Example 17: Modified Production Hosts Wherein the KDO Biosynthesis Route is Knocked Out for the Synthesis of LSTa

The production hosts as described in Example 15, wherein the genes encoding for a beta-1,3-GlcNAc transferase from Neisseria meningitidis (NmlgtA, SEQ ID NO: 16) and a beta-1,3-galactosyltransferase from E. coli 055:H7 (EcwbgO, SEQ ID NO: 18) are introduced in the genome, allowing the synthesis of the precursor LNT.

These organisms were cultured in the culture conditions supplemented with lactose as described in Example 1 and produced LSTa (a-NeuNAc-(2-3)-b-D-Gal-(1-3)-b-D-GlcNAc-(1-3)-b-D-Gal-(1-4)-Glc). Within the culture fluid no KDO-containing oligosaccharides were found.

### Example 18: Modified Production Hosts Wherein the KDO Biosynthesis Route is Knocked Out for the Synthesis of DSLNT

The production hosts as described in Example 15, wherein the genes encoding for a beta-1,3-GlcNAc transferase from Neisseria meningitidis (NmlgtA, SEQ ID NO: 16) and a beta-1,3-galactosyltransferase from E. coli 055:H7 (EcwbgO, SEQ ID NO: 18) are introduced in the genome, allowing the synthesis of the precursor LNT. Beside the α-2,3-sialyltransferase from Neisseria meningitidis (NmST, SEQ ID NO: 07), an extra α-2,6-sialyltransferase like ST6gall or ST6Galll was expressed on a plasmid and/or on the genome.

These organisms were cultured in the culture conditions supplemented with lactose as described in Example 1 and produced DSLNT (a-NeuNAc-(2-3)-b-Gal-(1-3)-[a-NeuNAc-(2-6)]-b-GlcNAc-(1-3)-b-Gal-(1-4)-Glc). Within the culture fluid no KDO-containing oligosaccharides were found.

### Example 19: Rendering Genes Less Functional

So called rendering genes less functional is a common practice in biotechnology. As described above there are several techniques to lower expression or render a gene less functional (such as the usage of siRNA, CrispR interference, RNAi, miRNA, asRNA, mutating genes, knocking-out genes, transposon mutagenesis, . . . ).

CrispR interference is one of the newest techniques in rendering genes less functional. It entails the design of an sgRNA that recognizes the gene of interest (the gene to be rendered less functional) and the expression of a mutated RNA-guided DNA endonuclease enzyme that lost its endonuclease activity (e.g., the dCas9 protein). The sgRNA is composed of a base pairing region mostly existing of 20 nucleotides downstream or upstream next to a PAM region (e.g., NGG in the case of dCas9). The base pairing region is complementary to a region into the target gene. The closer the base pairing region binds to the 5′ end of the gene target, the better the repression. To avoid off target repression, the base pairing region is BLASTed against the genome, ensuring there are no other regions complementary to the base pairing region apart from the gene of interest. An example of a design tool is described by Doench et. al. 2016 [Nature Biotechnology volume 34, pages 184-191(2016)] and is provided by most synthetic DNA providers.

Both dCas9 and the sgRNA are expressed in the cell according to the methods described in Example 1. Preferentially both are expressed from the genome, ensuring stable expression over several generations.

As an example, the kdsA gene with SEQ ID NO: 20 was rendered less functional by using the CrispRi technique described above. The used sgRNA was CCGCTCCTCCATCCACTCTTATCGTGGACC with TGG as a PAM sequence (nucleotides 186-215 of SEQ ID NO:20). The sequence was expressed by means of a constitutive promoter sequence (“PROM0012”) as described in Example 1. The expression cassette, together with dCas9, were introduced in the strains described in Example 2. In the resulting strains the KDO biosynthesis pathway was rendered less functional and hence no detectable KDO oligosaccharides are formed.

1. A microbial cell that naturally synthesizes keto-deoxyoctulosonate (KDO), which cell is genetically modified to produce an oligosaccharide, wherein KDO biosynthesis of the cell is knocked out or rendered less functional.

2. The cell according to claim 1, wherein the cell comprises at least one glycosyltransferase with affinity for cytidine 5′-monophospho-3-deoxy-d-manno-2-octulosonic acid (CMP-KDO).

3. The cell according to claim 1, wherein the cell is capable of synthesizing a nucleotide sugar selected from the group consisting of guanosine diphosphate (GDP)-fucose, GDP-mannose, GDP-rhamnose, CMP-N-acetylneuraminic acid, CMP-N-glycolylneuraminic acid, uridine diphosphate (UDP)-glucose, deoxythymidine diphosphate (dTDP)-glucose, UDP-galactose, UDP-N-acetylmannosamine, UDP-N-acetylglucosamine, UDP-N-acetylgalactosamine, UDP-glucuronic acid, UDP-xylose, UDP-arabinose, and UDP-galacturonic acid.

4. The cell according to claim 1, wherein:
an adenosine triphosphate (ATP)-dependent translocator encoding gene is overexpressed;
an inner membrane protein encoding gene is overexpressed;
a lauroyl acyltransferase encoding gene is overexpressed;
an endogenous ATP-dependent translocator encoding gene is modified; and/or
an endogenous inner membrane protein encoding gene is modified.

5. The cell according to claim 1, wherein the cell comprises a KDO-independent lauroyl acyltransferase encoding gene and/or wherein the cell comprises an adenosine triphosphate (ATP)-binding cassette multidrug transporter encoding gene.

6. The cell according to claim 4, wherein the ATP-dependent translocator, the inner membrane protein and/or lauroyl acyltransferase is overexpressed by an overexpression of the endogenous gene encoding the ATP-dependent translocator, inner membrane protein and/or lauroyl acyltransferase or by introducing and expressing the ATP-dependent translocator, inner membrane protein and/or lauroyl acyltransferase.

7. The cell according to claim 4, wherein the endogenous ATP-dependent translocator encoding gene is modified and/or the endogenous inner membrane protein encoding gene is modified as a point-mutation.

8. The cell according to claim 4, wherein:
the ATP-dependent translocator has 80% or more sequence identity to SEQ ID NO: 4 and has ATP-dependent translocator activity;
the inner membrane protein has 80% or more sequence identity to SEQ ID NO: 21 and has transmembrane transporter activity; and/or
the lauroyl acyltransferase has 80% or more sequence identity to SEQ ID NO: 1 and has lauroyl acyltransferase activity.

9. The cell according to claim 5, wherein the KDO-independent lauroyl acyltransferase encoding gene and/or the ATP-binding cassette multidrug transporter is i) introduced and expressed or ii) overexpressed in the cell.

10. The cell according to claim 5, wherein the cell expresses a gene encoding the KDO-independent lauroyl acyltransferase of SEQ ID NO: 2 or SEQ ID NO: 3, or a protein having at least 80% sequence identity thereto and having KDO-independent lauroyl acyltransferase activity.

11. The cell according to claim 5, wherein the cell expresses a gene encoding the ATP-binding cassette multidrug transporter of SEQ ID NO: 22, or a protein having at least 80% sequence identity thereto and having transmembrane transporter activity.

12. The cell according to claim 1, comprising at least one gene selected from the group consisting of genes encoding for D-arabinose 5-phosphate isomerase, 3-deoxy-D-manno-octulosonate 8-phosphate synthase, 3-deoxy-D-manno-octulosonate 8-phosphate phosphatase, and 3-deoxy-manno-octulosonate cytidylyltransferase, which is rendered less functional or knocked out.

13. The cell according to claim 1, wherein the cell produces a neutral, sialylated and/or fucosylated oligosaccharide.

14. The cell according to claim 1, wherein the cell produces a mammalian milk oligosaccharide.

15. The cell according to claim 2, wherein the glycosyltransferase is a sialyltransferase.

16. The cell according to claim 1, wherein the cell comprises a deleted or inactivated endogenous beta-galactosidase gene.

17. The cell according to claim 16, wherein the deleted or inactivated beta-galactosidase gene comprises E. coli lacZ gene.

18. The cell according to claim 1, wherein the microbial cell further comprises a deleted, inactivated, or mutated galactoside-O-acyltransferase (lacA) encoding gene.

19. The cell according to claim 1, wherein the cell further comprises a polynucleotide encoding at least one of the following additional proteins: an exporter protein or a permease exporting the synthesized oligosaccharide from the microbial cell.

20. The cell according to claim 1, wherein the cell is further genetically modified to contain a polynucleotide encoding a glycosidase for degrading interfering oligosaccharides, intermediates, side products or endogenous oligosaccharides generated by bacterial host cell, wherein the expression of the glycosidase is under control of a regulatory sequence.

21. The microbial cell according to claim 1, which is isolated.

22. A method of using the cell according to claim 1, the method comprising using the cell to produce sialylated oligosaccharides substantially free of KDO-lactose and/or KDO-oligosaccharide.

23. A method for fermentative production of an oligosaccharide substantially free of KDO-oligosaccharide, the method comprising:
cultivating a cell in favorable growing conditions, wherein the cell is the microbial cell of claim 1; and
optionally separating or isolating the oligosaccharide from the culture.

24. A method for producing a sialylated oligosaccharide by fermentation through genetically modified microbial cell naturally synthesizing keto-deoxyoctulosonate (KDO), the method comprising the steps of:
a) obtaining a microbial cell that naturally synthesizes KDO and is able to produce sialylated oligosaccharides and expressing a sialyltransferase with affinity for cytidine 5′-monophospho-3-deoxy-d-manno-2-octulosonic acid (CMP-KDO), and wherein the KDO-biosynthesis route of the cell is knocked out or rendered less functional;
b) culturing the cell from step a) in favorable growing conditions, thus producing the sialylated oligosaccharides; and
c) optionally, separating or isolating the sialylated oligosaccharide from the culture medium.

25. The method of claim 24, wherein the culture medium comprises at least one precursor for the production of the oligosaccharide.

26. The method of claim 25, wherein the cell produces the precursor internally.

27. The method according to claim 24, wherein the sialylated oligosaccharide is 3′-sialyllactose, 6′-sialyllactose, disialyl lacto-N-tetraose, sialylated lacto-N-triose, sialylated lacto-N-tetraose, sialylated lacto-N-neotetraose, 3-fucosyl-3′-sialyllactose, lacto-N-sialylpentaose LSTa, LSTb, LSTc, or LSTd.

28. The microbial cell of claim 1, wherein the cell is a Gram-negative microbial cell.

29. The method according to claim 24, wherein the oligosaccharide is isolated from the culture medium by means of unit operation selected from the group consisting of centrifugation, filtration, microfiltration, ultrafiltration, nanofiltration, ion exchange, electrodialysis, chromatography, simulated moving bed chromatography, simulated moving bed ion exchange, evaporation, precipitation, crystallization, spray drying and any combination thereof.

30. (canceled)

31. The method of claim 23, wherein the culture comprises at least one precursor for the production of the oligosaccharide.

32. The method of claim 31, wherein the cell produces the precursor internally.

33. The method according to claim 23, wherein the oligosaccharide is isolated from the culture in a manner selected from the group consisting of centrifugation, filtration, microfiltration, ultrafiltration, nanofiltration, ion exchange, electrodialysis, chromatography, simulated moving bed chromatography, simulated moving bed ion exchange, evaporation, precipitation, crystallization, spray drying, and any combination thereof.

34. The microbial cell according to claim 13, wherein the sialylated oligosaccharide is 3′-sialyllactose, 6′-sialyllactose, disialyl lacto-N-tetraose, sialylated lacto-N-triose, sialylated lacto-N-tetraose, sialylated lacto-N-neotetraose, 3-fucosyl-3′-sialyllactose, lacto-N-sialylpentaose LSTa, LSTb, LSTc, or LSTd.


SEQUENCE LISTING (58 sequences)

SEQ ID NO: 1 - protein, 306 aa, Escherichia coli K12 MG1655
MTNLPKFSTALLHPRYWLTWLGIGVLWLVVQLPYPVIYRLGCGLGKLALRFMKRRAKIVHRNLELCFPEMSEQERRKMVVKNFESVGMGLMETGMAWFWPDRRIARWTEVIGMEHIRDVQAQKRGILLVGIHFLTLELGARQFGMQEPGIGVYRPNDNPLIDWLQTWGRLRSNKSMLDRKDLKGMIKALKKGEVVWYAPDHDYGPRSSVFVPLFAVEQAATTTGTWMLARMSGACLVPFVPRRKPDGKGYQLIMLPPECSPPLDDAETTAAWMNKVVEKCIMMAPEQYMWLHRRFKTRPEGVPSRY

SEQ ID NO: 2 - protein, 307 aa, Francisella tularensis subsp. novicida
MNNNKFSPKNWGIWIVVGIMNCGSKLPLFTHKYIVLTIGLIIKPFLKSRNDIAYENLKIAFPEKSNKEIKKLVKKSYYSMVLSGAETTAAWFLSKKRFNKIEFKWEEGSRERFEKYHNDPDKKLIILGFHFHCIEIIGRYMGQEFPPFTVMYQKNGNDLIEDLIKEYREKSLYKCLDSKNFVSVIKSLKKGYTMWYAPDQDFGLESTGLENSVFAPFFGKLCSTLTVTPWLAQKTGAVVIPAYYVREKCLKKYKIVTGEPFEFIGDAYKDAEMTNKFLEDAVRKYPEQYLWQHRRYRTRPNGEPQIY

SEQ ID NO: 3 - protein, 269 aa, Francisella tularensis subsp. novicida
MLMRLAISIGFLAKPLLKKRQQIAITNLKIAFPEKSDQEIKKLADASYKSASMAGFESLISWFMTKKNFNKIPFDNDLRSFDEIHYDKDKTLLVLGFHFHCLEIAGRYIGENYSPFTVMYQYHNNPLMEYIITSSRKKYVTECFQRKNIIAVIKSLKRKVSMWYAPDQDFREHVVFAPFFGKLCTTLTVTPWLAEKTNATVIPAYYVRKPDLSGYSIITGEPIIFSGNDLEDAKLTNKILEEIIKKHPEQYLWQHRRYKTRPPGEEKIY

SEQ ID NO: 4 - protein, 582 aa, Escherichia coli K12 MG1655
MHNDKDLSTWQTFRRLWPTIAPFKAGLIVAGVALILNAASDTFMLSLLKPLLDDGFGKTDRSVLVWMPLVVIGLMILRGITSYVSSYCISWVSGKVVMTMRRRLFGHMMGMPVSFFDKQSTGTLLSRITYDSEQVASSSSGALITVVREGASIIGLFIMMFYYSWQLSIILIVLAPIVSIAIRVVSKRFRNISKNMQNTMGQVTTSAEQMLKGHKEVLIFGGQEVETKRFDKVSNRMRLQGMKMVSASSISDPIIQLIASLALAFVLYAASFPSVMDSLTAGTITVVFSSMIALMRPLKSLTNVNAQFQRGMAACQTLFTILDSEQEKDEGKRVIERATGDVEFRNVTFTYPGRDVPALRNINLKIPAGKTVALVGRSGSGKSTIASLITRFYDIDEGEILMDGHDLREYTLASLRNQVALVSQNVHLFNDTVANNIAYARTEQYSREQIEEAARMAYAMDFINKMDNGLDTVIGENGVLLSGGQRQRIAIARALLRDSPILILDEATSALDTESERAIQAALDELQKNRTSLVIAHRLSTIEKADEIVVVEDGVIVERGTHNDLLEHRGVYAQLHKMQFGQ

SEQ ID NO: 5 - protein, 228 aa, Neisseria meningitidis
MEKQNIAVILARQNSKGLPLKNLRKMNGISLLGHTINAAISSKCFDRIIVSTDGGLIAEEAKNFGVEVVLRPAELASDTASSISGVIHALETIGSNSGTVTLLQPTSPLRTGAHIREAFSLFDEKIKGSVVSACPMEHHPLKTLLQINNGEYAPMRHLSDLEQPRQQLPQAFRPNGAIYINDTASLIANNCFFIAPTKLYIMSHQDSIDIDTELDLQQAENILNHKES

SEQ ID NO: 6 - protein, 498 aa, Photobacterium sp. JT-ISH-224
MSEENTQSIIKNDINKTIIDEEYVNLEPINQSNISFTKHSWVQTCGTQQLLTEQNKESISLSVVAPRLDDDEKYCFDFNGVSNKGEKYITKVTLNVVAPSLEVYVDHASLPTLQQLMDIIKSEEENPTAQRYIAWGRIVPTDEQMKELNITSFALINNHTPADLVQEIVKQAQTKHRLNVKLSSNTAHSFDNLVPILKELNSFNNVTVTNIDLYDDGSAEYVNLYNWRDTLNKTDNLKIGKDYLEDVINGINEDTSNTGTSSVYNWQKLYPANYHFLRKDYLTLEPSLHELRDYIGDSLKQMQWDGFKKFNSKQQELFLSIVNFDKQKLQNEYNSSNLPNFVFTGTTVWAGNHEREYYAKQQINVINNAINESSPHYLGNSYDLFFKGHPGGGIINTLIMQNYPSMVDIPSKISFEVLMMTDMLPDAVAGIASSLYFTIPAEKIKFIVFTSTETITDRETALRSPLVQVMIKLGIVKEENVLFWADLPNCETGVCIAV

SEQ ID NO: 7 - protein, 371 aa, Neisseria meningitidis
MGLKKACLTVLCLIVFCFGIFYTFDRVNQGERNAVSLLKEKLFNEEGEPVNLIFCYTILQMKVAERIMAQHPGERFYVVLMSENRNEKYDYYFNQIKDKAERAYFFHLPYGLNKSFNFIPTMAELKVKSMLLPKVKRIYLASLEKVSIAAFLSTYPDAEIKTFDDGTGNLIQSSSYLGDEFSVNGTIKRNFARMMIGDWSIAKTRNASDEHYTIFKGLKNIMDDGRRKMTYLPLFDASELKTGDETGGTVRILLGSPDKEMKEISEKAAKNFKIQYVAPHPRQTYGLSGVTTLNSPYVIEDYILREIKKNPHTRYEIYTFFSGAALTMKDFPNVHVYALKPASLPEDYWLKPVYALFTQSGIPILTFDDKN

SEQ ID NO: 8 - protein, 417 aa, Escherichia coli K12 MG1655 [sequence omitted]

SEQ ID NO: 9 - protein, 415 aa, Escherichia coli W
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLVKLLHAIEVPLCVISVFKYSVANFDKRLSSTIFLIGFQIASSLGIVLLSTPTGILFDHAGYQTVFFAISGIVCLMLLFGIFFLSKKREQIVMETPVPSAI

SEQ ID NO: 10 - protein, 504 aa, Bifidobacterium adolescentis [sequence omitted]

SEQ ID NO: 11 - protein, 301 aa, Zymomonas mobilis
MKNDKKIYGCIEGGGTKFMLALIDSDRKMLAVERVPTTTPEETLGKRVEFFKKALPQYADSFASFGIASFGPLCLDRKSPKWGYITNTPKPFWPNTDVVTPFKEAFGCPVEIDTDVNGAALAENFWGASKGTHTSVYVTVGTGFGGGVLIDGKPIHGLAHPEMGHGIPIRHPDDRDFEGCCPYHGGCYEGLASGTAIRKRWGKALNEMEPAEFEKAREIIAFYLAHFNVTLQAFISPERIVFGGGVMHVDGMLASVRRQTAEIANSYFEGADFEKIIVLPGLGDQAGMMGAFALALAAENK

SEQ ID NO: 12 - protein, 609 aa, Escherichia coli K12 MG1655
MCGIVGAIAQRDVAEILLEGLRRLEYRGYDSAGLAVVDTEGHMTRLRRLGKVQMLAQAAEEHPLHGGTGIAHTRWATHGEPSEVNAHPHVSEHIVVVHNGIIENHEPLREELKARGYTFVSETDTEVIAHLVNWELKQGGTLREAVLRAIPQLRGAYGTVIMDSRHPDTLLAARSGSPLVIGLGMGENFIASDQLALLPVTRRFIFLEEGDIAEITRRSVNIFDKTGAEVKRQDIESNLQYDAGDKGIYCHYMQKEIYEQPNAIKNTLTGRISHGQVDLSELGPNADELLSKVEHIQILACGTSYNSGMVSRYWFESLAGIPCDVEIASEFRYRKSAVRRNSLMITLSQSGETADTLAGLRLSKELGYLGSLAICNVPGSSLVRESDLALMTNAGTEIGVASTKAFTTQLTVLLMLVAKLSRLKGLDASIEHDIVHGLQALPSRIEQMLSQDKRIEALAEDFSDKHHALFLSRGDQYPIALEGALKLKEISYIHAEAYAAGELKHGPLALIDADMPVIVVAPNNELLEKLKSNIEEVRARGGQLYVFADQDAGFVSSDNMHIIEMPHVEEVIAPIFYTVPLQLLAYHVALIKGTDVDQPRNLAKSVTVE

SEQ ID NO: 13 - protein, 159 aa, Saccharomyces cerevisiae
MSLPDGFYIRRMEEGDLEQVTETLKVLTTVGTITPESFSKLIKYWNEATVWNDNEDKKIMQYNPMVIVDKRTETVAATGNIIIERKIIHELGLCGHIEDIAVNSKYQGQGLGKLLIDQLVTIGFDYGCYKIILDCDEKNVKFYEKCGFSNAGVEMQIRK

SEQ ID NO: 14 - protein, 421 aa, Bacteroides ovatus
MDSKNNIGHSADISLTAELPIPIYNGNTIMDFKKLASLYKDELLDNVLPFWLEHSQDHEYGGYFTCLDREGKVFDTDKFIWLQSREVWMFSMLYNKVEKRQEWLDCAIQGGEFLKKYGHDGNYNWYFSLDRSGRPLVEPYNIFSYTFATMAFGQLSLTTGNQEYADIAKKTFDIILSKVDNPKGRWNKLHPGTRNLKNFALPMILCNLALEIEHLLDETYLRETMDTCIHEVMEVFYRPELGGIIVENVDIDGNLVDCFEGRQVTPGHAIEAMWFIMDLGKRLNRPELIEKAKETTLTMLNYGWDKQYGGIYYFMDRNGCPPQQLEWDQKLWWVHIETLISLLKGYQLTGDKKCLEWFEKVHDYTWEHFKDKEYPEWYGYLNRRGEVLLPLKGGKWKGCFHVPRGLYQCWKTLEEIKNIVS

SEQ ID NO: 15 - protein, 346 aa, Campylobacter jejuni
MKEIKIQNIIISEEKAPLVVPEIGINHNGSLELAKIMVDAAFSAGAKIIKHQTHIVEDEMSKAAKKVIPGNAKISIYEIMQKCALDYKDELALKEYTEKLGLVYLSTPFSRAGANRLEDMGVSAFKIGSGECNNYPLIKHIAAFKKPMIVSTGMNSIESIKPTVKILLDNEIPFVLMHTTNLYPTPHNLVRLNAMLELKKEFSCMVGLSDHTTDNLACLGAVVLGACVLERHFTDSMHRSGPDIVCSMDTKALKELIIQSEQMAIIRGNNESKKAAKQEQVTIDFAFASVVSIKDIKKGEVLSMDNIWVKRPGLGGISAAEFENILGKKALRDIENDAQLSYEDFA

SEQ ID NO: 16 - protein, 348 aa, Neisseria meningitidis
MPSEAFRRHRAYRENKLQPLVSVLICAYNVEKYFAQSLAAVVNQTWCNLDILIVDDGSTDGTLAIAQRFQEQDGRIKILAQAQNSGLIPSLNIGLDELAKSGMGEYIARTDADDIAAPDWIEKIVGEMEKDRSIIAMGAWLEVLSEEKDGNRLARHHEHGKIWKKPTRHEDIADFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTLPAGVWLDFAANGRMRRLFTLRQYFGILHRLLKNR

SEQ ID NO: 17 - protein, 275 aa, Neisseria meningitidis
MQNHVISLASAAERRAHIADTFGRHGIPFQFFDALMPSERLEQAMAELVPGLSAHPYLSGVEKACFMSHAVLWKQALDEGLPYITVFEDDVLLGEGAEKFLAEDAWLQERFDPDTAFIVRLETMFMHVLTSPSGVADYCGRAFPLLESEHWGTAGYIISRKAMRFFLDRFAALPPEGLHPVDLMMFSDFFDREGMPVCQLNPALCAQELHYAKFHDQNSALGSLIEHDRLLNRKQQRRDSPANTFKHRLIRALTKISREREKRRQRREQFIVPFQ

SEQ ID NO: 18 - protein, 265 aa, Escherichia coli O55H7
MIIDEAESAESTHPVVSVILPVNKKNPFLDEAINSILSQTFSSFEIIIVANCCTDDFYNELKHKVNDKIKLIRTNIAYLPYSLNKAIDLSNGEFIARMDSDDISHPDRFTKQVDFLKNNPYVDVVGTNAIFIDDKGREINKTKLPEENLDIVKNLPYKCCIVHPSVMFRKKVIASIGGYMFSNYSEDYELWNRLSLAKIKFQNLPEYLFYYRLHEGQSTAKKNLYMVMVNDLVIKMKCFFLTGNINYLFGGIRTIASFIYCKYIK

SEQ ID NO: 19 - protein, 373 aa, Campylobacter jejuni
MVKKILFITGSRADYSKIKSLMYRVQNSSEFELYIFATGMHLSKNFGYTVKELYKNGFKNIYEFINYDKYYQTDKALATTIDGFSRYANELKPDLIVVHGDRIEPLAAAIVGALNNILVAHIEGGEISGTIDDSLRHAISKLAHIHLVNDEFAKRRLMQLGEDEKSIFIIGSPDLELLNDNKISLSEAKKYYDINYENYALLMFHPVTTEITSIKNQADNLVKALIQSNKNYIVIYPNNDLGFELILQSYEEFKNNPRFKLFPSLRFEYFITLLKNADFIIGNSSCILKEALYLKTAGILVGSRQNGRLGNENTLKVNANSDEILKAINTIHKKQDLFSAKLEILDSSKLFFEYLQSGDFFKLSTQKVFKDIK

SEQ ID NO: 20 - DNA, 855 bp, Escherichia coli K12 MG1655
atgaaacaaaaagtggttagcattggcgacatcaacgtagcaaatgacctgccgttcgtactgtttggcggtatgaacgtgttggaatctcgcgatctggcgatgcgcatttgcgagcactacgtaactgtgacccagaaactgggtatcccttacgtgttcaaagcctcttttgacaaagccaaccgctcctccatccactcttatcgtggaccgggcctggaagaagggatgaaaatcttccaggagttgaagcagacttttggcgtgaaaattatcaccgacgttcacgaaccaagtcaggcacagcccgttgctgatgtcgtggatgtgattcagttgccggcgtttcttgctcgccagactgacctggttgaagccatggcgaaaaccggtgcggtaattaacgtcaagaaaccacagtttgtcagcccgggacagatgggtaatatcgttgataaattcaaagaaggcggcaacgaaaaagtgattctttgcgatcgcggtgctaacttcggctatgacaacctggttgtcgatatgctgggcttcagcattatgaagaaagtgtctggtaactcgccggtgattttcgacgtgacccacgcactgcaatgccgcgatccgtttggcgcagcttccggtggtcgtcgtgctcaggtggctgagctggcacgagccggtatggcggtaggtctggcggggctgtttattgaagcgcatccggatccggaacatgcgaaatgtgatggtccatccgcgctgccgctggctaaactggaaccgttcctcaagcagatgaaagcgattgatgatctggtgaaaggtttcgaagaactggataccagcaagtaa

SEQ ID NO: 21 - protein, 337 aa, Escherichia coli K12 MG1655
MTQENEIKRPIQDLEHEPIKPLDNSEKGSKVSQALETVTTTAEKVQRQPVIAHLIRATERFNDRLGNQFGAAITYFSFLSMIPILMVSFAAGGFVLASHPMLLQDIFDKILQNISDPTLAATLKNTINTAVQQRTTVGLVGLAVALYSGINWMGNLREAIRAQSRDVWERSPQDQEKFWVKYLRDFISLIGLLIALIVTLSITSVAGSAQQMIISALHLNSIEWLKPTWRLIGLAISIFANYLLFFWIFWRLPRHRPRKKALIRGTFLAAIGFEVIKIVMTYTLPSLMKSPSGAAFGSVLGLMAFFYFFARLTLFCAAWIATAEYKDDPRMPGKTQP

SEQ ID NO: 22 - protein, 590 aa, Lactococcus lactis subsp. lactis
MERGPQMANRIEGKAVDKTSIKHFIKLIRAAKPRYLFFIIGILAGIVGTLIQLQVPKMVQPLVNSFGHGVNGGKVALVIALYIGSAAVSAIAAIVLGIFGESVVKNLRTRVWDKMIHLPVKYFDEVKTGEMSSRLANDTTQVKNLIANSIPQAFTSILLLVGSIVFMLQMQWRLTLAMIIAVPVVMLIMFPIMTFGQKIGRTRQDSLANFQGIASESLSEIRLVKSSNAEKQASKKAENDVNALYKIGVKEAIFDGLMSPVMMLSMMLMIFGLLAYGIYLISTGVMSLGTLLGMMMYLMNLIGAVPTVATFFTELAKASGSTGRLTELLDEEQEVLHQGESLDLEGKTLSARHVDFAYDDSEQILRDISFEAQPNSIIAFAGPSGGGKSTIFSLLERFYQPTAGEITIDGQPIDNISLENWRSQIGFVSQDSAIMAGTIRENLTYGLEGDYTDEDLWQVLDLAFARSFVENMPDQLNTEVGERGVKISGGQRQRLAIARAFLRNPKILMLDEATASLDSESESMVQKALDSLMKGRTTLVIAHRLSTIVDADKIYFIEKGQITGSGKHNELVATHPLYAKYVSEQLTVGQ

SEQ ID NO: 23 - DNA, 70 bp, Artificial sequence
  primer - kdsA KO
attgtgctgcattaattaatcgacattttactcaagattaaggcgatcctccgcggtgcgggtgccaggg

SEQ ID NO: 24 - DNA, 76 bp, Artificial sequence
  primer - kdsA KO
atgaaaaaagtcttaacgcagaacgctaatactttatttttcaagcaaaaaagactacgcccccaactgagagaac

SEQ ID NO: 25 - DNA, 70 bp, Artificial sequence
  primer - kdsB KO
tgctggaaacggaagcccgcgtgctgactgctgatgagagtaaatcatgaccgcggtgcgggtgccaggg

SEQ ID NO: 26 - DNA, 72 bp, Artificial sequence
  primer - kdsB KO
taacggtacgacactcctcccaaaattggctgaagtgtcgtgaagtgaaactacgcccccaactgagagaac

SEQ ID NO: 27 - DNA, 70 bp, Artificial sequence
  primer - kdsC KO
catgatttactgcgtgcaggcgtagtgtaaagattcaaggataaacaacaccgcggtgcgggtgccaggg

SEQ ID NO: 28 - DNA, 72 bp, Artificial sequence
  primer - kdsC KO
agaaccgccagtgatagcacaatgataacccaacgtctggctttactcatctacgcccccaactgagagaac

SEQ ID NO: 29 - DNA, 70 bp, Artificial sequence
  primer - kdsD KO
gttgtactggttatcgccaatactcgttgaataactggaaacgcattatgccgcggtgcgggtgccaggg

SEQ ID NO: 30 - DNA, 72 bp, Artificial sequence
  primer - kdsD KO
tttgctcattgttgtttatccttgaatctttacactacgcctgcacgcagctacgcccccaactgagagaac

SEQ ID NO: 31 - DNA, 68 bp, Artificial sequence
  primer - gutQ KO
agagagcaatgagtgaagcactactgaacgcgggacgtcagacgttaatgccgcggtgcgggtgccag

SEQ ID NO: 32 - DNA, 69 bp, Artificial sequence
  primer - gutQ KO
cggctggcgaaacgtctgggattgaaggattaaataatcccggcctgatactacgcccccaactgagag

SEQ ID NO: 33 - DNA, 80 bp, Artificial sequence
  primer - msbA nt 52 CG to TA
gcatcgtctcaatctggtctcaaatgcataacgacaaagatctctctacgtggcagacattccgccgactgtggtcaacc

SEQ ID NO: 34 - DNA, 49 bp, Artificial sequence
  primer - msbA nt 52 CG to TA
tgccgtctcactaaggtctcacagcttattggccaaactgcattttgtg

SEQ ID NO: 35 - DNA, 50 bp, Artificial sequence
  primer - msbA nt 148 CG to TA
gcatcgtctcaatctggtctcaaatgcataacgacaaagatctctctacg

SEQ ID NO: 36 - DNA, 34 bp, Artificial sequence
  primer - msbA nt 148 CG to TA
tatgcgtctctgtgacttaaggagcgataacatg

SEQ ID NO: 37 - DNA, 34 bp, Artificial sequence
  primer - msbA nt 148 CG to TA
actacgtctcatcacttcttgatgatggctttgg

SEQ ID NO: 38 - DNA, 49 bp, Artificial sequence; identical to SEQ ID NO: 34
  primer - msbA nt 148 CG to TA

SEQ ID NO: 39 - DNA, 44 bp, Artificial sequence
  primer - yhjD nt 400 CG to TA
gcatcgtctcaatctggtctcaaatgacgcaggaaaacgagatc

SEQ ID NO: 40 - DNA, 34 bp, Artificial sequence
  primer - yhjD nt 400 CG to TA
tatgcgtctcttacactgctgaacggcggtgttg

SEQ ID NO: 41 - DNA, 33 bp, Artificial sequence
  primer - yhjD nt 400 CG to TA
actacgtctcatgtacgactgtagggcttgtcg

SEQ ID NO: 42 - DNA, 45 bp, Artificial sequence
  primer - yhjD nt 400 CG to TA
atgccgtctcactaaggtctcacagcttaaggctgcgttttcccc

SEQ ID NO: 43 - DNA, 20 bp, Artificial sequence
  primer - kdsA KO
tccggatggcgaaatttggc

SEQ ID NO: 44 - DNA, 20 bp, Artificial sequence
  primer - kdsA KO
tttactggcttcctggtggc

SEQ ID NO: 45 - DNA, 20 bp, Artificial sequence
  primer - kdsA KO
agatccctatgaaattcgcg

SEQ ID NO: 46 - DNA, 20 bp, Artificial sequence
  primer - kdsA KO
cgcatctggcactgtttgcc

SEQ ID NO: 47 - DNA, 20 bp, Artificial sequence
  primer - kdsB KO
ggtgacattaggatgctgcc

SEQ ID NO: 48 - DNA, 20 bp, Artificial sequence
  primer - kdsB KO
tctttgagttgttcccagcg

SEQ ID NO: 49 - DNA, 21 bp, Artificial sequence
  primer - kdsB KO
cgtctgcttgaaatcattgcc

SEQ ID NO: 50 - DNA, 20 bp, Artificial sequence
  primer - kdsB KO
aatggcttaattcggcacgc

SEQ ID NO: 51 - DNA, 20 bp, Artificial sequence
  primer - kdsC KO
atacgggcgatgagatcccg

SEQ ID NO: 52 - DNA, 20 bp, Artificial sequence
  primer - kdsC KO
ttctgggttatagacgagcg

SEQ ID NO: 53 - DNA, 20 bp, Artificial sequence
  primer - kdsC KO
tgaggcactgaacttaatgc

SEQ ID NO: 54 - DNA, 20 bp, Artificial sequence
  primer - kdsC KO
tgcgaagttgagagtctggc

SEQ ID NO: 55 - DNA, 21 bp, Artificial sequence
  primer - kdsD KO
gtgagtgatccccaatgtggc

SEQ ID NO: 56 - DNA, 20 bp, Artificial sequence
  primer - kdsD KO
cccgcgctgataacgccagg

SEQ ID NO: 57 - DNA, 19 bp, Artificial sequence
  primer - gutQ KO
ctgattgcagaagctgccg

SEQ ID NO: 58 - DNA, 19 bp, Artificial sequence
  primer - gutQ KO
gaagtggcggcggtgcccg